(19) United States
(12) Patent Application Publication
(10) Pub. No.: US 2026/0248420 A1
(43) Pub. Date: Aug. 27, 2026

(54) PHYSIOLOGICAL STATE DETECTION USING MULTI-ANALYTE IN VIVO SENSING

(71) Applicant: PercuSense, Inc., Valencia, CA (US)

(72) Inventors: Bradley LIANG, Bloomfield Hills, MI (US); Brian KANNARD, Los Angeles, CA (US)

(21) Appl. No.: 19/226,840

(22) Filed: Jun. 3, 2025

Related U.S. Application Data
(60) Provisional application No. 63/761,585, filed on Feb. 21, 2025.

(51) Int. Cl. A61B 5/1473; A61B 5/00; A61B 5/145; G16H 50/20

(52) U.S. Cl. CPC A61B 5/1473; A61B 5/0024; A61B 5/14532; A61B 5/14542; A61B 5/14546; A61B 5/4809; A61B 5/6846; A61B 5/7203; A61B 5/7225; A61B 5/7271; G16H 50/20; A61B 2560/0468

(57) ABSTRACT

A physiological state monitoring system includes an implantable multi-analyte sensor probe including a first working electrode configured to generate a first electrical signal indicating concentration of a first analyte and a second working electrode configured to generate a second electrical signal indicating a concentration of a second analyte. Monitor system control circuitry is configured to generate a feature vector that includes one or more of a baseline-subtracted analyte value, a first-order temporal derivative, a second-order temporal derivative, or an inter-analyte ratio based on the digital sensor data, access parameters of a machine-learning classification model trained on historical multi-analyte sensor logs and associated ground-truth physiological states, and use the machine-learning classification model to generate, based on the feature vector, a real-time classification label and an associated confidence value identifying a physiological state.

### RELATED APPLICATION(S)

This application claims the benefit of U.S. Provisional Patent Application Ser. No. 63/761,585, filed on Feb. 21, 2025, the complete disclosure of which is hereby incorporated by reference in its entirety.

### BACKGROUND

The present disclosure generally relates to the field of percutaneous analyte sensing for diagnostic purposes. Body-worn physiological monitors are increasingly used in clinical, military, and industrial settings to track the physiological condition of a subject outside of laboratory environments. Many existing systems, however, measure only a single analyte, such as glucose, or deliver results after a substantial delay, limiting their usefulness when rapid insight into a person's metabolic status or potential chemical exposure is required.

### SUMMARY

Described herein are methods, systems, and devices relating to the acquisition and interpretation of data from multiple biochemical markers in near real time so that a changing physiological state of a subject can be recognized promptly and communicated to appropriate personnel and/or devices. Modern medicine, defense, and occupational safety solutions often lack compact, continuously operating systems capable of translating multiple, simultaneously measured biochemical markers into a clear picture of a person's immediate physiological state. Whether the goal is to recognize early signs of chemical-agent exposure on the battlefield, detect opioid overdose in paramedic care, or monitor metabolic stress during athletic training, existing tools are generally single-analyte, point-in-time, and/or lab-bound, delivering results too narrow or too late for rapid intervention. Inventive concepts presented herein advantageously provide body-worn platforms that are configured to sample several key analytes in parallel, fuse such sensor streams with trained logic/model(s), and output an actionable, real-time assessment of the wearer's current physiologic or toxicologic condition.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silico, or overlaid on one or more of the physical components. Virtual elements can be presented on any combination of screens, headsets, holographically, projected, loudspeakers, headphones, pressure transducers, temperature transducers, or using any combination of suitable technologies.

For the purpose of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 shows a multi-analyte-based physiological condition monitoring system in accordance with one or more embodiments.

FIGS. 2A-2C are block diagrams of multi-analyte sensor probe configurations in accordance with one or more embodiments.

FIG. 3 is a data transformation/flow diagram illustrating systems and processes for generating physiological condition classifications based on multiple analyte sensor signals in accordance with one or more embodiments.

FIG. 4 is a data transformation/flow diagram illustrating systems and processes for generating physiological condition classifications using trained artificial intelligence model(s) in accordance with one or more embodiments.

FIG. 5 is a data transformation/flow diagram illustrating systems and processes for training artificial intelligence model(s) for physiological condition classification in accordance with one or more embodiments.

FIGS. 6A and 6B show example user interface elements in accordance with one or more embodiments.

FIG. 7 is an exemplary illustration of real-time glucose, lactate, oxygen, and ROS data for various physiological states such as sleep, exercise, a meal, and stress, in accordance with one or more embodiments.

FIGS. 8A, 8B, and 8C provide graphs showing example multi-analyte signal and opioid exposure correlations in accordance with one or more embodiments.

FIGS. 9A, 9B, 9C, and 9D provide graphs showing example multi-analyte signal and organophosphate exposure correlations in accordance with one or more embodiments.

FIGS. 10A and 10B provide graphs showing example multi-analyte signal and mustard agent exposure correlations in accordance with one or more embodiments.

FIGS. 11A and 11B provide exemplary views of an A-side and a B-side of an implantable sensor probe in accordance with one or more embodiments.

FIG. 11C is an exemplary cross-section illustration of an analyte sensor probe illustrating diffusion of analyte, reactant and reaction by-product in accordance with one or more embodiments.

FIGS. 12A and 12B provide exemplary views of an A-side and a B-side of an implantable sensor probe in accordance with one or more embodiments.

FIGS. 13A and 13B provide are exemplary views of an A-side and a B-side of an implantable sensor probe in accordance with one or more embodiments.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, various examples may be carried out in a manner that achieves or optimizes one advantage or group of advantages as taught herein without necessarily achieving other aspects or advantages as may also be taught or suggested herein.

Certain spatially relative terms, such as “outer,” “inner,” “upper,” “lower,” “below,” “above,” “vertical,” “horizontal,” “top,” “bottom,” “distal,” “proximal,” and similar terms, are used herein to describe a spatial relationship of one device/element or anatomical structure to another device/element or anatomical structure. It should be understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. Spatially relative terms, including those listed above, may be understood relative to a respective illustrated orientation of a referenced figure.

Certain reference numbers are reused across different figures of the figure set of the present disclosure as a matter of convenience for devices, components, systems, features, and/or modules having features that are similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

The present disclosure relates to systems, devices, and methods for monitoring physiological and/or chemical-exposure states of a subject using body-worn, multi-analyte sensing systems that feed real-time biochemical data into trained machine-learning models to classify and report the subject's current physiological or chemical-exposure state. Wearable and minimally invasive biosensing technologies can be used to continuously track individual biomarkers, such as glucose, lactate, tissue oxygen, ketones, choline, and the like. With respect to certain single-analyte monitoring solutions, such systems may fail to capture/detect complex physiological events that manifest when multiple biochemical pathways shift in concert. Furthermore, situations that demand rapid insight, such as chemical-agent exposure, opioid overdose, extreme metabolic distress, can be difficult to recognize early with systems that analyze only one biomarker at a time or require external laboratory processing. The present disclosure describes multi-analyte monitoring platforms that combine percutaneous or on-body sensor probes/arrays with data-processing architecture trained to interpret the collective behavior of several biomarkers. Signals acquired from the sensor probe can be processed/transformed through a feature-extraction pipeline and entered into one or more trained machine-learning models, such as random-forest, gradient-boosted-tree, or neural-network classifiers. The trained AI models can advantageously be configured to output real-time classifications indicative of the physiological or toxicological state of the subject/wearer and/or provide confidence metrics or supporting feature attributions. The physiological state classification results can be delivered to a local or remote user interface that can present status information, trigger alerts, or forward data for further action.

### Physiological State/Chemical Exposure Detection Systems

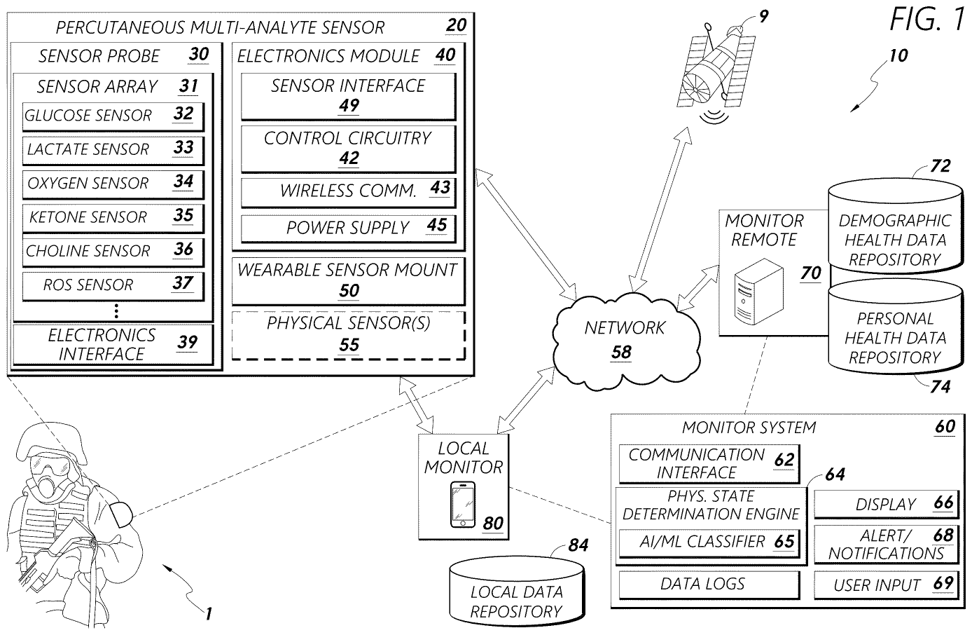

Certain examples are disclosed herein in the context of percutaneous sensor systems that integrate implantable sensing technology, on-body and/or local monitoring device(s), and/or remote servers, utilized together to facilitate real-time detection of physiological states and/or chemical exposures, and to provide actionable alerts to users. With respect to the framework and interrelationship of hardware, software, and data flow of such systems, FIG. 1 provides an exemplary block diagram showing components of a system 10 configured to detect and process signals and/or data sets indicative of analyte tissue concentrations of a subject 1, and automatically determine a physiological/exposure state of the subject 1, in accordance with certain implementations of the present disclosure.

The system 10 advantageously provides technical improvements in physiological state and chemical agent exposure determination/detection by integrating multi-analyte sensing with real-time state classification functionality, thereby providing heightened accuracy and reliability in identifying, e.g., hazardous exposures. Broadly, the system 10 includes a percutaneous multi-analyte sensor system 20 that includes a sensor probe 30 that is electrically coupled to an electronics module 40 via an electronics interface 39. The sensor probe 30 advantageously is configured to capture, when disposed in a transcutaneous/percutaneous position inserted at least partially into subcutaneous tissue of the subject/user 1, multiple analyte signals (e.g., glucose, oxygen, choline, ketones, reactive oxygen species (ROS), lactate, etc.) at a single insertion site. A wearable sensor mount 50 may be associated with the sensor system 20. For example, the sensor mount 50 may be a skin-mounted/mountable unit to which the sensor probe 30 and/or electronics module 40 is/are physically coupled when the sensor probe 30 is inserted in the subject's tissue. Collectively, the components of the sensor system 20 can provide a hardware-based platform capable of continuous, real-time measurements of a plurality of analytes for improved physiological state and/or chemical exposure detection.

In certain embodiments, the analyte sensor probe 30 is an electrochemical sensor probe that includes a sensor array 31 configured to measure/detect specific molecules of interest in vivo. Using specialized electrode configurations, the sensor array 31 can implement electrochemical sensing to simultaneously measure concentrations of glucose, oxygen, choline, and/or one or more additional analytes (e.g., lactate, ketones, ROS). For example, a glucose sensor 32 of the sensor array 31 can be configured to implement amperometric detection with a selective enzyme coating of glucose oxidase. In such implementations, the glucose oxidase enzyme may catalyze the oxidation of glucose to gluconic acid, producing hydrogen peroxide (H₂O₂) in proportion to the glucose concentration. An electrode then amperometrically detects the generated peroxide (or the associated oxygen consumption), with the resulting current serving as a quantitative measure of the glucose level. The choline sensor 36 can, similarly to glucose, implement an enzymatic approach, such as using choline oxidase. In such implementations, the choline oxidase enzyme may catalyze the oxidation of choline to betaine, producing hydrogen peroxide (H₂O₂) as a byproduct. The generated hydrogen peroxide can then be detected electrochemically (e.g., via an amperometric electrode), with the resulting current being proportional to the choline concentration. For oxygen sensing, the sensor array 31 can implement non-enzymatic, electrochemical (e.g., Clark electrode) or luminescence-based techniques to directly measure oxygen via electrochemical reduction or by monitoring changes in light emission. In some embodiments, the sensor array 31 can include additional sensors to detect or measure other molecules or analytes of interest such as, but not limited to lactate sensors, ketone sensors using potentiometric methods, reactive oxygen species (ROS) sensors using chronoamperometry, and/or sensors to detect/measure acetylcholine, alcohol and/or the like. Any such additional analyte sensors can be integrated to further enhance detection of chemical exposure conditions. The incorporation of three or more sensor channels in a single device enables improved accuracy in detecting and predicting chemical exposure and/or toxicity, supporting advanced feedback-based chemical exposure mitigation.

The sensor array 31 can additionally or alternatively include one or more lactate sensors 33, ketone sensors 35, and/or reactive oxygen species (ROS) sensors 37. It should be understood that sensor array implementations in accordance with the present disclosure can include any subset of the illustrated sensors, which may be collectively embodied on one or more sensor probes. For example, the sensor array 31 can include working-electrode sites on the probe configured to track lactate, β-hydroxybutyrate (ketone), and/or reactive oxygen species (ROS) in the same interstitial-fluid compartment as the glucose sensor 32, oxygen sensor 34, and/or choline sensor 36. With respect to the lactate sensor/channel 33, such sensor may employ lactate oxidase immobilized beneath a perm-selective membrane layer (e.g., comprised of material such as but not limited to polyurea, polyurethane, and/or silicone), wherein oxidation of lactate to pyruvate generates hydrogen peroxide that is amperometrically measured at a working electrode. For the ketone sensor 35, β-hydroxy-butyrate dehydrogenase may or may not be co-immobilized with an NAD⁺/osmium-polymer redox mediator so that the enzymatically produced NADH is re-oxidized at the working electrode, yielding a current proportional to β-hydroxybutyrate (BHB) concentration while minimizing interference from glucose. For the ROS sensor 37, a thin electropolymerized poly(ortho-phenylenediamine) film may be implemented that is doped with ferrocene derivatives, deposited on a separate microelectrode. Reactive oxygen species such as hydrogen peroxide and superoxide can modulate the redox state of the ferrocene, allowing the system to read out ROS burden via differential pulse voltammetry referenced to a shared counter electrode. Each sensor/channel of the sensor array 31 can advantageously be isolated by patterned diffusion-limiting membranes and individually addressed by the potentiostat array (or other analog front-end circuitry), enabling simultaneous, cross-talk-free measurement of the various analytes in real time.

The electronics interface 39 facilitates electrical communication between the analyte sensor probe 30 and the electronics module 40. While illustrated as part of the sensor probe 30, in some embodiments, the electronics interface 39 may be embodied at least in part in the electronics module 40 and/or sensor mount 50. As the electronics interface 39 is intended to interface between the analyte sensor probe 30 and the electronics module 40, its relative association or location between the elements or components within the sensor system 20 should not be construed as limiting.

In some embodiments, the electronics module 40 includes a sensor interface 49, a wireless communication module 43 (e.g., transceiver configured to transmit sensor date), and/or a power supply 45. The sensor interface 49 can be configured to enable electrical coupling between the electronics module 40 and the electronics interface 39 to allow signals generated by the analyte sensor probe 30 to be passed to the control circuitry 42 of the electronics module 40. The terms “circuitry” and “control circuitry” are used herein according to their broad and ordinary meanings, and may refer to any individual or collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Circuitry referenced herein may further comprise one or more storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in examples in which circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

The control circuitry 42 of the electronics module 40 may be configured to perform certain signal processing, amplification, filtering, conversion, calibration, and management/control functions for the sensor system 20. The control circuitry 42 can be utilized for real-time signal processing, including filtering, amplification, and transformation of raw electrochemical signals into calibrated glucose, oxygen, and/or choline concentrations. For example, the control circuitry 42 can be configured to implement adaptive algorithms to correct for temperature variations or cross-analyte interference, thereby promoting accurate real-time data for physiological/exposure state determination. By integrating these functions within a single hardware platform, the system 10 offers enhanced reliability and responsiveness, supporting improved safety and efficacy in physiological and/or chemical exposure state detection.

As described in detail herein, exposure of a human subject to mustard agents, opioids, and/or organophosphates can trigger respective physiological disruptions that can be reflected in tissue concentrations of lactate, choline, and/or oxygen levels. In addition, concentrations of glucose, oxygen, lactate, reactive oxygen species, and/or other analytes can indicate, individually or collectively, changes in a subject's physiological state (e.g., sleep, meal, exercise, stress conditions) that are relevant to the subject's health and/or treatment. In some implementations, the analyte signals that serve as the basis for physiological state determination/classification in accordance with systems disclosed herein can include or consist of glucose, lactate, and choline, or any other combination of the analytes listed above.

The control circuitry 42 can be configured to apply machine-learning models trained on personal, demographic, and/or historical data to dynamically adjust physiological state and/or chemical exposure assessment based on detected analyte trends. In some embodiments, the control circuitry 42 enables transformation of raw signals from the analyte sensor probe 30 to be representative of the respective molecule or analyte being detected. The control circuitry 42 can further generate and/or display information that is more meaningful for the subject than analyte or molecular concentrations, such as output indicating present or predicted physiological states or chemical exposure risks/levels.

The electronics module 40 further comprises a wireless communication module (e.g., transceiver) 43. The wireless communication module 43 enables communication between the sensor system 20 and other components within the system 10. In some embodiments, the communication module 43 supports two-way communication via one or more protocols, such as Wi-Fi (e.g., IEEE 802.11), Bluetooth (Classic or Low-Energy), satellite communication protocols, cellular standards (4G, 5G, and beyond), or any other suitable short-or long-range wireless technologies. The communication module 43 can enable data acquired by the sensor system 20 to be transmitted in either raw, partially processed, or fully processed to other components within the system 10. The communication module 43 can further enable data from other components within the system 10 to be used as input for the sensor system 20. For example, in some embodiments, the communication module 43 enables patient-related data from one or more data storage repositories 72, 74 to be dynamically input to the control circuitry 42 of the sensor system 20. A power supply 45 can also be included as part of the electronics module 40. In certain embodiments, the power supply 45 comprises an energy storage device such as a disposable or rechargeable battery. The power supply 45 additionally or alternatively may include one or more solar cells, capacitors, fuel cells, and the like.

In some embodiments, the sensor system 20 includes a wearable sensor mount 50, which may be attached or coupled to the subject/user 1 (e.g., using an adhesive patch disposed on a bottom surface of the skin-mountable sensor mount 50). The sensor mount 50 can be configured to receive and secure the electronics module 40. In some embodiments, the sensor probe 30 may further be coupled/attached to the sensor mount 50. Insertion of the sensor probe 30 may serve to couple the sensor mount 50 to the subject 1. Subsequent to insertion of the sensor probe 30, the electronics module 40 may be coupled to the sensor mount 50 and begin providing power to the sensor probe 30. In some embodiments, the electronics module 40 may be removably coupled to the sensor mount 50, such that the electronics module 40 may be reusable in its entirety independently of the sensor probe 30 and/or mount 50, which may be particularly advantageous for embodiments that utilize a rechargeable or replaceable power supply.

In some embodiments, one or more physical sensors 55 may be optionally included as part of the sensor system 20. The inclusion of physical sensor(s) 55 can enable detection of parameters that can affect the integrity or validity of data acquired via the sensor probe 30. Exemplary, non-limiting physical sensors that may be integrated within the sensor system 20 include, but are not limited to, accelerometers, thermal sensors, and the like, which can provide improvements in chemical exposure detection technologies by enhancing system accuracy through detection and response to external conditions that can influence analyte levels. For instance, accelerometer data can indicate periods of sustained motion corresponding to exercise or movement, allowing the system to account for the effects of motion/exercise on analyte levels, such as glucose.

The system 10 further includes one or more data repositories 72, 74, which may be remote from the sensor system 20 and accessible via a network 58. The data repositor(ies) 72, 74 can advantageously store data that can influence or have an impact on data provided by the sensor system 20. Exemplary data retained in the data repositor(ies) 72, 74 can include, but is not limited to, data indicating attributes of the subject 1 and/or demographic population(s) having related characteristics/classifications with respect to age, gender, height, weight, body mass index, waist circumference, blood pressure (diastolic or systolic), cholesterol, chronic and/or acute medication quantities, chronic conditions, and/or the like. In certain embodiments, health metric that may be recorded in an electronic or physical health record may be input and stored and accessed by one or more components of the system 10. The demographic 72 and/or personal 74 health data can enable the system to contextualize real-time sensor outputs, such as correlating analyte trends with metabolic health conditions (e.g., insulin resistance, non-alcoholic fatty liver disease, etc.) to refine state detection processes.

The demographic health data 72 and the personal health data 74 can be stored in the same physical data storage device(s) or server(s). Both the demographic data repository 72 and the personal data repository 74 may store data of a similar type. However, in certain embodiments, the demographic data repository 72 is anonymized, while the personal data repository 72 includes data indicating a specific connected user/subject. The demographic data repository 72 can enable analysis of data across various demographics represented by the data stored therein. For example, in some embodiments, the demographic data repository 72 enables trained artificial intelligence model(s) to analyze the data for patterns or trends that can be applied to modify or control other components within the system 10.

A network 58 is included within the system 10 to enable communication between various components within the system 10. The network 58 may leverage various communication protocol(s), such as cellular or mobile networks (e.g., 5G, 4G and the like), Wi-Fi, Bluetooth, LoRaWAN, Zigbee and/or the like. The network 58 can enable data from the sensor system 20 to be stored in the data repository 72. Additionally, the network 58 can enable the use of data stored in the demographic data repository 72 and/or personal health data repository 74 as input to control or modify control of other components within the system 10.

In some embodiments, the system 10 includes a monitor system 60 that handles data reception, processing, display, and/or storage associated with multi-analyte sensor data and physiological state classifications. As used herein, the terms “physiological state” and “physiological state classification” can refer to any objectively detectable condition or mode of function of a living subject, including but not limited to toxicological states such as exposure to exogenous chemical agents or pharmaceuticals; metabolic states associated with nutrient intake, digestion, absorption, or endogenous glucose-ketone balance; activity-related states such as rest, sleep stages, light or strenuous exercise, or recovery; homeostatic or stress states characterized by thermoregulatory load, hormonal response, hydration level, or oxidative stress; and any combination or transition among the foregoing that can be inferred from one or more measured biochemical, biophysical, or contextual signals.

The monitor system 60 may reside or be embodied locally (e.g., as a smartphone or wearable controller) and/or remotely (e.g., as a server or command center), depending on the particular implementation or use case. By providing real-time data visibility, classification analytics, and alerts, the monitor system 60 can help facilitate prompt detection of changing physiological state and/or chemical exposures, and subsequent user or medical response. The monitor subsystem 60 may be configured to perform various physiological state management functionality, such as data collection, processing, analysis, and/or presentation of sensor-derived information within the system 10. The monitor system 60 can be instantiated on any suitable computing platform, whether on-premises, wearable, handheld, or remote/cloud-based.

The monitor system 60 includes communication interface circuitry 62, which may be utilized for data reception and/or communication management. The communication interface 62 can receive input from the multi-analyte sensor 20, directly or indirectly, via wired or wireless protocols (e.g., Bluetooth, Wi-Fi, LoRa, cellular, satellite). The communication interface 62 may advantageously support secure channels, encrypting data in transit to protect sensitive physiological readings. In some implementations, the monitor system 60 is configured to synthesize/utilize data from multiple biosensors and/or data stores, allowing for aggregation of real-time and historical information. The communication interface 62 can be configured to filter and smooth incoming sensor signals, compute baseline or rolling statistics, and/or extract relevant features for chemical exposure detection.

The monitor system 60 includes a physiological state determination engine 64, which may comprise circuitry configured to use the sensor data as input to an artificial intelligence (AI) state classification engine 65. The classification engine 65 can be configured to implement one or more processes to determine if the subject 1 has experienced a chemical exposure event or other physiological state change, such process(es) utilizing threshold-based heuristics, advanced machine-learning pipelines (e.g., gradient-boosted decision trees, neural networks), or the like. In some implementations, the physiological state detection engine 64 is configured to implement multi-analyte integration, wherein a combination of lactate, choline, oxygen, and/or other analyte sensor readings/data are processed to enable the system 60 to make exposure determinations between multiple agent types (e.g., organophosphates, mustard agents, opioids). Additionally or alternatively, the engine 64 can use glucose, lactate, oxygen, and/or reactive oxygen species sensor input to make state determinations between multiple possible state classes (e.g., sleep, exercise, stress, meal digestion).

The monitor system 60 can include one or more displays 66 or other output components. The system 60 can be configured to generate graphical user interface data representative of real-time analyte levels, historical trends, and/or exposure/state classification determinations, which may be rendered at least in part on the display(s) 66. The system 60 can further comprise one or more alert/notification devices/circuitry 68 configured to generate and/or output alerts in one or more forms, such as audio signals, vibration, push notifications, textual prompts, or other output designed to alert a user and/or prompt immediate action or further investigation. In some implementations, the system 10 can include user input circuitry/component(s) 69, which may be part of the communication interface 62, for allowing a user or operator to adjust thresholds, sensitivity levels, notification preferences (e.g., silent mode vs. high-priority alarms), or other settings of the system 60.

The monitor system 60 can include data storage, which may be embodied in any of the data repositories shown in the system 10, for maintains logs of sensor readings, classification results, and/or user interactions. Such records can be accessed for classification input, post-event analysis, training refinement, or other purposes. In some implementations, for personal health records, demographic baselines, or other useful data, the monitor system 10 can query specialized repositories, such as to retrieve population-level reference data or personalized baseline statistics, which may be maintained locally or accessed from a remote server, for improved detection accuracy. The monitor system 60 may advantageously encrypt stored data and enforce role-based access controls to protect sensitive information.

The physiological state determination/detection engine 64 can be configured to track performance metrics (e.g., detection accuracy, false positives), and utilize such feedback for adaptive improvements to the trained detection model(s). For example, the artificial intelligence (AI) and/or machine learning (ML) model(s)/circuitry 65 can be adaptively retrained or otherwise updated based on newly acquired data, adjusting classification weights and/or logic to account for evolving patient readings/response and/or broader demographic populations. In some embodiments, the monitor system 60 can interface with various remote server APIs or data formats, enabling third-party integration or external system coordination (e.g., sending alerts to a centralized emergency management platform). Whether implemented on a local device 80 (e.g., smartphone, wearable controller) or in a remote environment 70 (e.g., server, cloud platform), or both, the monitor system 60 can perform the same core functionalities described above. In some embodiments, a single physical device may handle all tasks (e.g., preprocessing, classification, alerting, etc.), while in others, tasks may be distributed across multiple computational layers/devices.

An example local monitor 80 is shown, which may perform some or all of the functionality of the monitor system 60, depending on the implementation. The local monitor 80 may be implemented as a smartphone, tablet, dedicated handheld device, or other portable computing device equipped with properly configured control circuitry (e.g., microprocessor, microcontroller, SoC) and data storage 84 to store sensor readings, classification rules, temporary logs, and/or the like.

The local monitor 80 maintains wireless connectivity (e.g., Bluetooth, Wi-Fi, LoRa, cellular, satellite) with the implantable/on-body sensor system 20. In some implementations, the local monitor 80 is configured for two-way communication with the remote monitor 70 (e.g., server) through a secure network connection. The local monitor 80 may receive raw or partially processed biosensor signals and/or feature vectors from the implant device 20, and may perform certain on-board data processing, such as data smoothing, feature extraction, and/or classification (e.g., generate local exposure/analyte classification inference). In some embodiments, the local monitor 80 caches data temporarily for local processing and/or relay data to the remote monitor 70 for cloud-based analytics.

The local monitor 80 may include one or more visual electronic displays and/or other output mechanism(s), which may be used to present real-time analyte readings (e.g., lactate, choline, oxygen) as numeric values or trend curves/profiles. Such output may display exposure alerts when a chemical agent classification is triggered, optionally providing recommended actions or automated emergency notifications. In some embodiments, the local monitor 80 supports user modification of calibration settings, threshold customization, or manual data labeling. The local monitor 80 may retain in the local data repository 84 short-to medium-term historical logs for post-event analysis. By directly coupling a local monitor 80 with the implant sensor 20, the system 10 can provide low-latency state/exposure alerts regardless of network availability.

The remote monitor 70 may likewise be configured to implement any of the functionality of the monitor system 60 and/or local monitor 80. In some implementations, whereas the local monitor 80 may provide immediate, on-the-spot alerts and/or moderate data processing for minimal latency, the remote monitor 70 may perform more complex or big-data operations, such as large-scale algorithm training, cross-user pattern detection, and/or population-level baselining. For example, in some implementations, the local and/or remote monitor may aggregate information from multiple subjects, possibly along with physical location information, in order to improve the accuracy and timeliness of physiological state classification algorithms. Additionally, multi-subject information may be used by the monitoring system to determine geographical boundaries of environmental exposure. In situations where network connectivity is unavailable or intermittent, the local monitor 80 may queue data for upload when a stable connection resumes. In some implementations, when the remote system 70 is unreachable, the local monitor 80 may be configured to provide core detection and alerts using locally-stored trained models.

For military applications, as an example, the sensor 20 and/or monitor system 60 can be configured to wirelessly communicate using secure, military-grade network(s) or protocol(s). For example, the wireless communication module 43 and/or communication interface 62 can be configured to transmit data using encrypted radio frequency (RF) links, frequency-hopping, and/or spread spectrum techniques to avoid jamming or interception, examples including Joint Tactical Radio System (JTRS) or other standard military protocols. The system 10 may further utilize satellite communications with satellites 9 (e.g., military satellites or commercial satellites) providing secure encryption layers for beyond-line-of-sight communications. In some implementations, the wireless communication module 43 and/or communication interface 62 can be configured to implement mesh networking or ad hoc networking, such as military MANET (Mobile Ad Hoc Network) protocols, which can allow devices to form a secure peer-to-peer network with dynamic routing. In some examples, the wireless communication module 43 and/or communication interface 62 can be configured to communicate using dedicated tactical data links (e.g., Link 16, Link 22, or other NATO STANAG (Standardization Agreement) networks). In some examples, the wireless communication module 43 and/or communication interface 62 can be configured to utilize encrypted IP-based systems, such as secure VPN tunnels over public or private IP networks, using high-grade cryptographic protocols (e.g., IPsec with NSA Suite B or other MIL-SPEC encryption).

In some examples, the wireless communication module 43 and/or communication interface 62 can be configured to operate over standard cellular networks, but with end-to-end encryption and additional security layers. In some examples, the control circuitry 42 of the sensor system 20 includes tamper resistance, secure crypto modules, and/or hardware-based key management to guard against cyber threats. The wireless communication module 43 and/or communication interface 62 can further be configured to interface to existing command-and-control software, letting field commanders monitor soldier biometrics in real time. Although certain communication protocols are described, it should be understood that components of the system 10 can use any secure protocol or network, including, but not limited to, one or more tactical radio networks, satellite links, or any secure IP-based transport.

### Sensor Probe Configurations for Chemical Exposure Detection

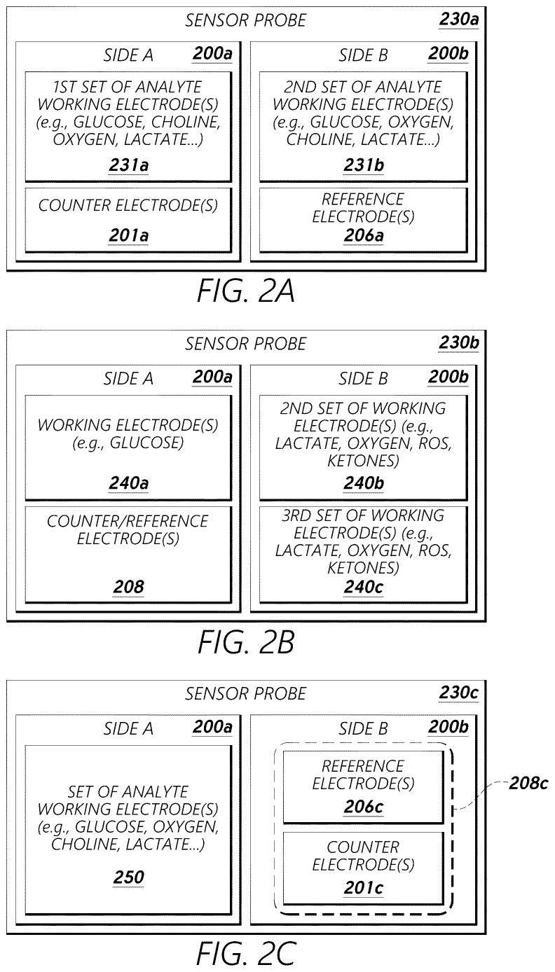

In the context of system 10 described above, a central component is the multi-analyte sensor 20 configured to measure various physiological markers (e.g., choline, lactate, oxygen, glucose) from a single implantable or percutaneous probe. As discussed, integrating multiple sensing functions onto one substrate/probe can enable comprehensive monitoring within a compact footprint. FIGS. 2A-2C illustrate exemplary arrangements of electrode probe 230a-230c arrangements, which may be implemented on a thin, elongated sensor substrate having first (‘A-side’) and second (‘B-side’) faces. In some embodiments, two-electrode systems (working electrode plus a combined counter-reference electrode) are employed, while in others, three-electrode systems (working, counter, and reference) are implemented. Electrodes may be placed on one or both faces of the substrate, with each electrode tailored to detect a particular analyte or serve as a counter/reference element. It will be understood that the arrangements shown are exemplary in nature and can be modified or combined with additional electrode structures or sensing chemistries without departing from the inventive concepts presented herein. Any one or more of the sensor probes 230a-230cshown in these figures may be implemented in the system 10 described above to detect and measure different analytes such as glucose, lactate, oxygen, and/or choline, among others.

Each illustrated sensor probe (e.g., 230a-230c) comprises two primary faces described herein an ‘A-side’ 200a and a ‘B-side’ 200b. For example, a multi-analyte sensor probe may have a thin, elongated body with a rectangular cross-section, producing two relatively large, flat faces on opposite sides. The edges of the elongate probe may run lengthwise, connecting the two faces and providing narrower surface areas. Depending on the intended use and the targeted analyte(s), one or both faces may have electrochemical electrodes associated therewith.

The electrode configurations herein can include either a two-electrode system or a three-electrode system (or a hybrid), enabling multiple analytes to be measured from a single, minimally invasive probe. For example, in a two-electrode system, working electrode(s) (e.g., one working electrode for each analyte measured) of the probe may operate in connection with a combined counter-reference electrode. In a three-electrode system, the working electrode(s) may operate in connection with separate counter and reference electrodes. Multiple working electrodes can share a single reference or counter electrode when monitoring more than one analyte.

FIG. 2A illustrates a three-electrode sensor probe configuration 230a, in which the A-side 200a comprises one or more working electrodes 231a (e.g., one or more of glucose, oxygen, lactate, ketones (e.g., β-hydroxybutyrate), reactive oxygen species (ROS), and/or choline working electrodes) and one or more counter electrodes 201a. The B-side 200b is configured with a second set of one or more working electrodes 231b for detecting one or more different or similar analytes (e.g., one or more of glucose, lactate, ketones, ROS, oxygen, and/or choline working electrodes) and further includes one or more reference electrodes 206a. This arrangement allows each analyte to be measured independently, while still sharing a common reference or counter as needed for stable electrochemical measurements. The second set of electrode(s) 231b may include electrodes for sensing one or more analytes not sensed by the first set of electrode(s) 231a, and vice-versa.

FIG. 2B illustrates a two-electrode sensor probe configuration 230b, in which the A-side 200a includes a first set of one or more working electrodes 240a (e.g., one or more of glucose, oxygen, lactate, ketones, ROS, and/or choline working electrodes) and a combined counter and reference electrode 208. The B-side 200b includes a second set of one or more working electrodes 240b for detecting one or more different or similar analytes (e.g., one or more of glucose, oxygen, lactate, ketones, ROS, and/or choline working electrodes), as well as a third set of one or more working electrode(s) 240c for detecting one or more different or similar analytes (e.g., one or more of glucose, oxygen, lactate, ketones, ROS, and/or choline working electrodes). The different sets of working electrode(s) can detect the same analyte (for redundancy) or different analytes. In some implementations, the first, second, and third sets of working electrode(s) may be configured to detect glucose, lactate, and choline, or glucose, lactate, and oxygen, in any order or configuration. By utilizing a two-electrode system, the design of FIG. 2B can provide a sensor probe with a reduced number of required electrodes on each face, simplifying manufacturing while still permitting robust multi-analyte sensing.

FIG. 2C shows another three-electrode sensor probe configuration 230c, wherein the A-side 200a includes one or more working electrodes 250 (e.g., one or more of glucose, oxygen, lactate, ketones, ROS, and/or choline working electrodes). For example, the set of analyte working electrodes 250 can include a glucose-sensing working electrode, an oxygen-sensing working electrode, and a choline-sensing working electrode or other combination. The B-side 200b contains one or more reference electrodes 206c and one or more counter electrodes 201c. Positioning multiple working electrodes (for different analytes) on the same face/side can simplify wiring and/or bonding processes, while the reference and counter electrodes on the opposite face can maintain stable potentials and current flow. While shown separately in FIG. 2C, the reference 206c and counter 201c electrodes may be combined in a single counter/reference electrode 208c on Side B 200b.

The configurations illustrated in FIGS. 2A-2C are exemplary rather than limiting. Some implementations may combine two-electrode and three-electrode systems on the same probe. For instance, a portion of the probe can have a working electrode paired with a combined counter-reference electrode for a first set of one or more analytes, while another section of the probe can feature a full three-electrode system with a second set of one or more analytes. Similarly, multiple working electrodes on one side can share counter or reference electrodes on the other, depending on the electrochemical requirements of each analyte.

In various embodiments, the precise positioning of electrodes is strategically chosen to maximize measurement accuracy and minimize interference among analytes. For instance, lactate (or glucose) electrode(s) may be placed near the distal tip of the sensor probe to ensure exposure to interstitial fluid under higher metabolic demand. Meanwhile, choline and/or oxygen electrodes may be located slightly more proximal or on the opposite side, spaced apart to avoid electrochemical cross-talk. A reference electrode might be positioned near the center on a respective side, equidistant from multiple working electrodes, thereby enhancing potential stability for all measurements. The counter electrode can be placed at an opposing end to ensure uniform current distribution. Such arrangements can help ensure accurate, real-time detection of multiple analytes, such as glucose, choline, and oxygen, from a single implant while maintaining stable signals and reducing cross-interference.

In some implementations, glucose-and lactate-sensing working electrodes can be placed near the distal tip of the sensor probe, where interstitial fluid is most metabolically active, while a ketone-sensing electrode is positioned just proximal to one or both of those sites so that it samples the same micro-environment without necessarily sharing an immediate diffusion layer. Choline-and/or oxygen-sensing electrodes may be disposed on the opposite major face of the sensor probe, or along a staggered longitudinal track to avoid direct redox interference. In examples where reactive oxygen species (ROS) sensing is implemented, such electrode (e.g., microelectrode), which may require only picoampere-scale currents, may be embedded mid-shaft and shielded by insulating trenches to isolate its relatively sensitive redox layer. A shared reference electrode can advantageously be located near the geometric center of the probe, equidistant from some or all of the working electrodes, to maintain a stable potential for the various analyte channels, and a counter electrode is situated at the proximal end to promote uniform current return across the sensor array. Such spatial arrangements can permit simultaneous, real-time detection of multiple biochemical markers from a single minimally invasive implant, thereby enabling robust determinations of chemical-exposure states or broader physiological conditions while minimizing patient discomfort and maximizing signal fidelity. The diverse electrode layouts as described herein can provide flexible configurations to capture vital biochemical data from multiple analytes, enabling robust detection of analyte levels and exposure to chemical agents while minimizing patient discomfort and maximizing measurement accuracy.

### Exposure Detection System Operation

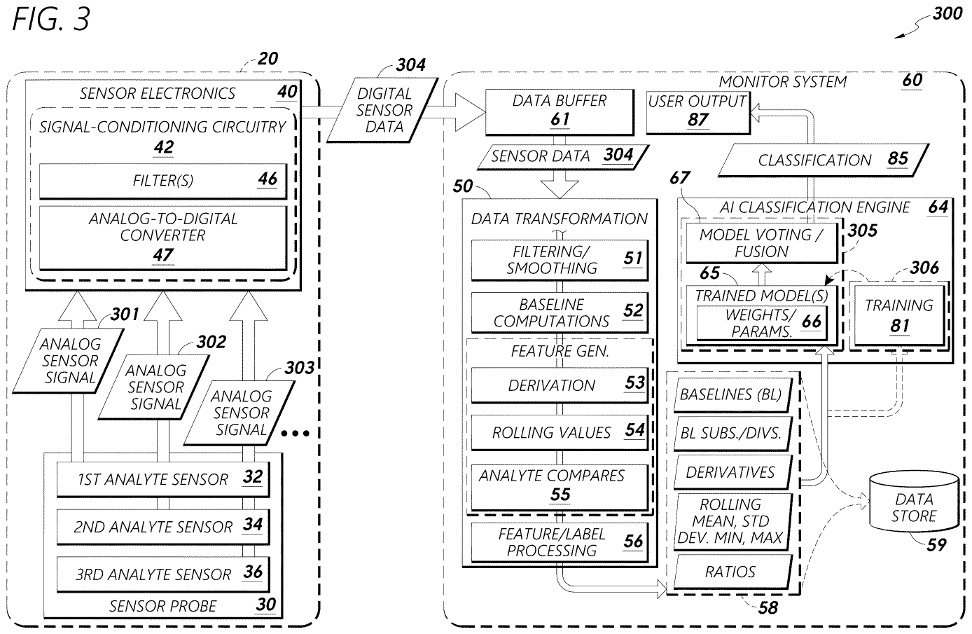

The above description provides details of broader system architectures and physical layouts associated multi-analyte sensors. Operation of a system including such features can involve the transport and transformation of sensor-related signals and data structures to produce physiological state determinations/output (e.g., chemical exposure detection), which can be implemented, at least in part, by the monitor system 60 and/or other component(s) of the system 10 of FIG. 1. FIG. 3 illustrates a block and dataflow diagram for a chemical exposure detection system 300 in accordance with one or more embodiments. The system 300 is configured to implement data acquisition, processing, feature engineering, and/or classification functionality, utilizing one or more trained AI models/pipelines. The dataflow represented in the system 300 reflects the transformation of raw data into data structures/packets that indicate real-time detection of changes in physiological state (e.g., sleep or exercise states, or hazardous exposures).

Sensor data acquisition can be performed using a sensor probe 30, which may be a component of a multi-analyte sensor system 20, as described above. The sensor probe hardware 30 can comprises multiple sensor electrodes for multi-analyte biosensor functionality. In the illustrated example, the sensor probe includes at least a first analyte (e.g., lactate or glucose) sensor electrode 32/33, a second analyte (e.g., oxygen) sensor electrode 34, and a third analyte (e.g., choline) sensor electrode 36, although it should be understood that any number or combination of analyte sensors may be implemented in the system 300 among at least the group of sensors including glucose, lactate, oxygen, ketones, reactive oxygen species (ROS), and/or choline sensors. During operation, the sensor probe 30 and associated sensors may be embedded in tissue of a user/subject for sensing analyte concentrations therein. Each of the sensors produces analog signals proportional to the respective analyte's concentration and/or electrochemical current. For example, FIG. 3, shows analog sensor signals for at least the first (e.g., glucose) 301, second (e.g., oxygen) 302, and third (e.g., choline) 303 signals provided by the sensor probe 30, which may be received and conditioned by an electronics module 40 component of the sensor system 20.

The electronics module 40 includes certain signal-conditioning control circuitry 42. The signal-conditioning circuitry 42 can include an analog-to-digital converter 47 and/or other front-end circuitry configured to convert the incoming analog signals 301, 302, 303 into digital form. The circuitry 42 may further include initial hardware-based filtering circuitry 46 (e.g., low-pass/band-pass filters) configured to, for example, attenuate high-frequency noise before data transmission. Digitized sensor data 304 may be transmitted to a local or remote computing platform (e.g., a smartphone, wearable processor, or server) configured to operate as the monitor system 60. Such data transmission may advantageously be performed using secure wireless (Bluetooth, LoRa, Wi-Fi, satellite) or wired interfaces.

The digital/digitized sensor data 304 generated by the sensor electronics 40 can be transmitted to a monitor system 60, which may be embodied in whole or in part in one or more local and/or remote computing devices/systems. The monitor system 60 is configured to refine and transform the raw sensor readings represented by the sensor data 304 into feature-rich data structures for analyte monitoring and/or chemical exposure detection. The monitoring system 60 can load the sensor data/samples 304 into data storage 61 serving a buffering function, wherein buffered sensor data may be stored in connection with timestamps data to facilitate chronological alignment across multiple sensors/analytes (e.g., lactate, choline, oxygen).

The sensor data 304, or some form of data based at least partially thereon, can be provided to certain data transformation/control circuitry 50 of the monitor system 60. The data transformation circuitry 50 can be implemented as a feature-generation engine, and can include, for example, data filtering and/or smoothing circuitry 51 configured to implement a short, sliding window (e.g., 3-5 samples) to scan the sensor data to reduce or eliminate spikes or single-sample outliers, which may be due to transient noise or physical motion artifacts. In some implementations, the data transformation circuitry and/or one or more modules thereof 51-58 can be embodied, generated, and/or otherwise achieved and/or implemented as/using digital circuitry/code (e.g., in firmware/software). The circuitry 51 can further apply a decay factor to older data points to create a smoothed signal, wherein rapid oscillations are dampened while gradual trends remain clear. The utilization of the filtering/smoothing circuitry can mitigate random fluctuations and isolate genuine sensor signal behaviors, such that subsequent data transforms capture actual changes in analyte levels rather than anomalies.

The data transformation circuitry 50 can further include baseline computation circuitry 52 configured to generate rolling baseline signals/data-structures (e.g., 1-hour or 2-hour rolling median) for one or more of the sensor channels. The system 60 may store such baseline data in a baseline repository of the system data storage 59 for reference in subsequent operations to detect relative changes over time. In some implementations, the rolling baseline signal for one or more sensor channels (e.g., lactate, choline, oxygen) is compared to the smoothed signal. The baseline may be computed over a longer window (e.g., 1 or 2 hours) to reflect the user's physiological steady state. Together, the filtering/ smoothing 51 and baseline computation 52 circuitry can beneficially transform sensor signals to remove noise and highlight physiologically relevant changes.

With the raw sensor data having been filtered and smoothed, the system 60 can generate certain transformed signals and features that better capture meaningful physiological changes indicative of, e.g., chemical exposure. For example, the data transformation circuitry 50 can further include signal derivation/translation circuitry 53 configured to generate new derived and/or transformed signals/data-points indicating how sensor readings change over time and/or in relation to each other, rather than focusing solely on absolute values. The derivation/translation circuitry 53 can determine patterns relating to rates at which analyte levels rise or fall, degrees of difference between sensor signals, and/or how the sensor readings compare to a user's typical (baseline) state/profile. For example, sudden jumps or drops in sensor readings can be weighted higher than slow, steady movements and may be an early sign of exposure.

The derivation/translation circuitry 53 may be configured to generate baseline-subtracted signals/values (e.g., indicating the difference between present sensor signal(s)/value(s) and baseline signal(s)/value(s), as well as baseline-divided signals (e.g., indicating present sensor signal(s)/value(s) divided by baseline signal(s)/value(s)). Subtracting the baseline can produce a signal that highlights acute deviations from normal conditions rather than absolute sensor readings. Rapid or sustained shifts in the baseline-subtracted signal can reveal early onset of an exposure event, which might otherwise be obscured by individual variability. With regard to the baseline-divided signals, such signals/data express changes as a relative proportion or percentage of the user's normal reading. This can be especially useful where users'absolute sensor values differ due to metabolic or anatomical factors (e.g., certain individuals might have naturally higher lactate baselines). A ratio above or below certain thresholds (e.g., 1.2×baseline) may provide a more robust trigger for physiological state and/or chemical exposure detection than an absolute difference.

The data derivation/transformation circuitry 53 can further be configured to generate first- and/or second-order derivative signal(s)/value(s). Such derivative signals/values can provide an approximation of the rate of change (first-order) and/or the rate of acceleration (second-order) in the analyte sensor signals/readings over time. The derivative signals can be generated using finite difference coefficients on the smoothed sensor data, or other finite difference approximations. A high first-order derivative signals a rapid rise or drop, potentially reflecting the body's immediate physiological response to activity or toxins (e.g., precipitous drop in choline upon organophosphate exposure). The second-order derivative highlights acceleration or deceleration in those changes, providing an early-warning if the rate is speeding up (e.g., an exposure quickly intensifying). Together, the features/data generated by the derivation/transformation circuitry 53 can reveal non-obvious relationships among multiple analytes, enabling advanced detection of physiological state/exposure events.

The data transformation circuitry 50 further includes a module/circuitry 54 for generating rolling sensor reading values including aggregate statistics over, for example, relatively short rolling windows (e.g., 30 or 60 minutes) to capture local trends and variability. Examples of rolling values generated by the circuitry 54 can include average, standard deviation, minimum, or maximum values over past 30 or 60 minute periods for a given analyte signal. Such data points can be used to indicate intra-hour fluctuations, revealing slow-building changes/exposures or consistent anomalies. Standard deviation values can indicate volatility in sensor readings, which can trigger certain responses, as sustained high volatility can reflect an evolving physiological state/exposure scenario, whereas stable readings are more likely normal.

In some implementations, the data transformation circuitry 50 is configured to generate data/signals based on comparisons between analyte readings from different sensor electrodes of the multi-analyte sensor system 20. For example, the data transformation circuitry 50 can include a module/circuitry 55 configured to generate ratios and/or differences between readings of separate sensors, such as among glucose, ketones, lactate, choline, and/or oxygen sensors. The analyte compare/ratio data structures/points can indicate or trigger detection of certain chemical exposure or physiological state events that characteristically alter, or correlate with, the balance among analytes more prominently than, or in a manner different from, how they alter or correlate with a single analyte alone. For instance, an organophosphate might strongly depress choline but have a lesser effect on oxygen. In some implementations, the system 60 may trigger organophosphate detection and/or responsive action based on a choline/oxygen ratio, which may highlight that choline is dropping disproportionately. Differences between analyte readings can be leveraged to detect immediate synergy or divergence in signals, such as where lactate is rising quickly while oxygen remains constant. A difference signal indicating rising lactate with more constant oxygen may be determined as indicating a state where the subject's metabolism is shifting toward anaerobic pathways without a commensurate drop in overall oxygen, which can be associated with organophosphate exposure, which can trigger muscle overactivity or other metabolic stress while not immediately impairing oxygen supply. Therefore, the system 60 may respond to such analyte comparison by trigging an output or responsive action associated with exposure to certain nerve agents or other toxins that disrupt normal aerobic processes. Physiologically, a similar signature (increasing lactate with constant O₂) can indicate intense exertion or localized tissue dysfunction.

In addition to ratios and differences, the circuitry 55 may compare pairs of sensor signals by applying a rolling window (e.g., 10 samples) to generate Pearson correlation coefficients, which can provide a measure of linear correlation between two sensor signals (e.g., with a range from −1 to +1). The circuitry 55 can further be configured generate mutual information between two (or more) different sensor signals, such as using discretization into, e.g., 10 bins. Such mutual information generation can involve converting each continuous sensor signal (e.g., oxygen, ketones, glucose, choline, lactate, etc.) into a discrete variable by splitting its numerical range into a predetermined number (e.g., 10) of intervals (or “bins”). Each data point can be assigned to one of these bins based on its value. Once both sensor signals are transformed in this way, the joint frequency distribution of the two binned variables can be measured, and mutual information that indicates/quantifies an amount of data that is shared by the two sensor signals, indicating how much data of one sensor reduces uncertainty about the other, and/or vice versa. Mutual information generation can provide data indicating relationships that might not be purely linear (unlike correlation). The circuitry 55 may “bin” the continuous sensor signals to provide discrete probability distributions (e.g., by building histograms and/or compute probabilities). If the presence of a certain bin range in one signal corresponds strongly to a specific bin range in the other signal, the mutual information value generated by the circuitry 55 will generally be high, indicating that such signals have a shared pattern relevant to detecting physiological state change/event or chemical exposures. By applying multiple-bin discretization, the system 60 can meaningfully determine how changes in one sensor track or diverge from changes in another, even if those relationships are not straightforward or linear. In terms of example use cases, some chemical exposures might decouple two analytes that usually track together (e.g., oxygen and choline might diverge after specific nerve agent exposure). The system 60 may therefore identify a drop in correlation between two sensor signals as an early marker of abnormal physiology. Together, the correlation, mutual information, and derivative features generation implemented by the data transformation circuitry 50 can beneficially transform the sensor data to reveal non-obvious relationships among multiple analytes, enabling advanced detection of exposure and/or physiological state events.

The data transformation circuitry 50 may include further module(s) 56 configured to perform certain feature and/or label processing functionality. Such circuitry 56 can be configured to implement feature normalization, wherein one or more of the transformed data 58 outputs of the various modules of the data transformation circuitry 50 are scaled and/or normalized (e.g., via min-max or z-score), such that varying sensor ranges (e.g., lactate in mM vs. oxygen in mmHg) do not skew downstream classification. In some implementations, the module 56 can toggle normalization on or off depending on calibration outcomes or real-time performance metrics. The circuitry 56 can further implement event labeling and/or alignment, wherein sensor data timestamps are matched with known exposure windows or ground-truth logs. For example, each data sample may be assigned a classification label (e.g., “mustard,” “opioid,” “organophosphate,” “exercise,” “meal,” “sleep,” “none,” etc.). When no chemical exposure or noteworthy physiological state is recorded, intervals may be labeled accordingly to provide robust, non-event training examples. The circuitry 56 may further be configured to automatically filter out early-stage sensor readings during an initial stabilization period. For example, newly implanted or activated sensors often exhibit noisy drift; excluding these data points can prevent artificial anomalies from influencing the subsequent models.

The system 300 further includes an AI classification engine 64, which includes circuitry trained on a data set for transforming one or more of the output data structures 58 from the data transformation circuitry 50 into output labels that indicate a physiological state and/or chemical exposure state of the subject. The feature set 58 may be passed, in whole or in part, along an AI classification path 305 and/or a training path 306, depending on the nature of the underlying data. Although examples are presented herein in the context of analyte levels/signals being analyzed for classification functionality of disclosed systems, it should be understood that any analyte processing/analysis disclosed herein can be used to generate to implement/deploy real-time regression models on multi-analyte sensor data in order to estimate analyte concentrations for physiological state management.

The trained AI model(s) 65 can be configured to implement one or more machine-learning models, which can have any suitable or desirable trained model framework(s). For example, the model(s) 65 may comprise a random forest model configured to combine the output of multiple decision trees to determine a classification label/result. In some implementations, the model(s) may comprise a gradient-boosting model (e.g., eXtreme Gradient Boosting (XGBoost) model) designed to use a gradient boosting mechanism/logic, with advanced feature importance and high accuracy for structured sensor data. Other possible model(s) include a feed-forward neural network, which may be designed to implement a multi-layer perceptron architecture with dropout and regularization suitable for handling complex nonlinear relationships. Outputs from the models may be combined in some manner by a model voting/fusion module/circuitry 67.

The AI classification engine 64 can operate using one or more sets of trained weights/parameters, the values of which may be predetermined by a training engine/module 81 for transforming input data to classification outputs. The AI classification engine 64 can receive one or more features of the normalized feature set 58 in real time, or near real time. The model(s) 65 can assign labels indicating classification 85 based on detection/determination of one or more physiological and/or chemical-exposure states of the subject associated with the input data, such as “meal,” “ketosis,” “exercise,” “sleep,” “organophosphate exposure,” “mustard agent exposure,” “opioid exposure,” “no exposure,” or other relevant classes, indicating whether the sensor data correlates with a known physiological state and/or exposure profile. In some embodiments, the AI model(s) 65 can output a confidence measure or probability associated with each classification for improved interpretability and risk assessment. The monitor system 60 may re-train or update the AI classifier model(s) 65 using the training module 81 based on newly aggregated data and deploys updated model weights/parameters 66, ensuring that the system 60 remains accurate across various conditions and potential new chemical threats.

The local data repository 59 may be used to store recent sensor readings, derived features, classification outcomes, device status logs, and/or the like. The classification engine 64 and/or data transformation module 50 may be configured to aggregate classification results with raw or partially-processed data to generate comprehensive historical logs for model improvement, recalibration, or offline analytics.

The system 300 may include user output signal/data generator circuitry 87, which may be configured generate signals/data that cause/trigger user interface presentation or other output (e.g., audible signals, haptic output, etc.) indicative of real-time physiological/exposure classifications. In some implementations, the output generator circuitry 87 is configured to generate graphical user interface data or other output signals/data representing or indicating notifications or alarms upon detection of potential exposures, wherein such notifications/alarms may provide an indication of the nature of the physiological/exposure state that is the basis of the output. The monitor system 60 may further be configured to communicate exposure events to a command center or remote monitoring platform for coordinated response and/or user safety tracking.

The system 300 advantageously addresses real-world hazards (e.g., chemical exposure) with a specific, inventive arrangements of hardware, including multi-analyte sensor devices and data-processing circuitry, and specialized AI. The system 300 improves upon existing biosensor frameworks by enabling multi-analyte, real-time classification with minimal user calibration. As illustrated by FIG. 3, the system 300 implements a multi-stage data flow architecture, from raw sensor readings through noise removal, feature engineering, event labeling, and finally AI classification, resulting in immediate and actionable determinations of physiological state and/or chemical exposure. Unlike mere data analysis software, the system 300 is inextricably tied to specialized sensor hardware and performs specific data transformations that yield crucial, real-time detection of physiological states and/or hazardous agents with high sensitivity and specificity.

### Classification Model Architecture

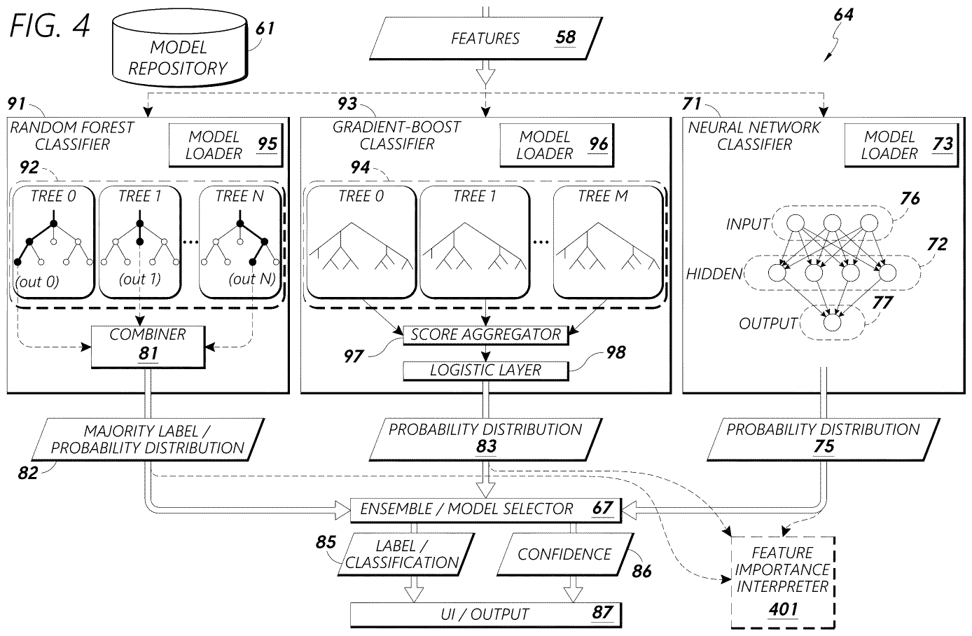

The above description provides details of a monitor system including AI classification engine circuitry for classification of multi-analyte sensor data from a multi-analyte sensor device implanted in a user/subject. FIG. 4 illustrates a block and dataflow diagram for an AI classification system 64 in accordance with one or more embodiments. The system 64 is configured to preform trained-model-based classification for detecting physiological states and/or chemical exposures of a subject. The system 64 can be configured to implement multiple classifiers in real time, and provides technologically integrated solutions for detecting physiological states and/or chemical exposures. The classification engine 64 can run on a wearable microcontroller, a smartphone app, and/or a cloud server, at least in part. Real-time constraints (e.g., sub-1-minute detection) may favor on-device or near-edge classification system embodiment.

FIG. 3 described in detail above illustrates how a feature vector/set may be generated for input to the AI classification engine 64. The data structure(s) 58 can comprise a consolidated representation of a sensor time slice (or sample) that is provided to the classification engine 64. For example, rather than feeding the classifier the raw sensor values 304 from the multi-analyte sensor 20 (e.g., unfiltered glucose, ketone, choline, lactate, and/or oxygen readings), the system 300 first processes and transforms the data through various stages using feature/data extraction and/or transformation circuitry 50 configured to implement certain filtering, baseline subtraction, derivative calculation, pairwise ratios, correlation, and/or other transformation/extraction operations to produce the feature set 58. In some implementations, the processed signals and/or derived data structures/metrics 58 can be assembled into a single, ordered list of numerical values, which may be considered a feature vector. For example, the feature data set 58 may include baseline-subtracted analyte values, first-order and/or second-order derivative values, rolling analyte values, mutual information between two or more analyte values, pairwise ratios of two or more analyte values, etc. Collectively, these data can provide an analyte-based snapshot that machine-learning model(s) (e.g., Random Forest, XGBoost, and/or Neural Network) can use to detect/determine a physiological state and/or chemical exposure condition of a subject, and if so, a character/type thereof.

The classification engine 64 can comprise one or more classifier modules, which can be implemented, managed, and/or combined for robust, low-latency detection. In some embodiments, the system 64 may run a single classifier (e.g., XGBoost), such as if prior evaluation shows that model alone satisfies performance requirements. Alternatively, multiple classifiers (random forest, XGBoost, and/or neural network) can operate in parallel, wherein their outputs may be combined by a voting or confidence-weighted scheme. The system 64 may maintain certain model configurations/parameters in data storage 61. For example, hyperparameters associated with one or more classifiers (e.g., tree depth, learning rate, dropout rate) can be loaded from a configuration file, or can be set dynamically based on real-time performance metrics. Such a modular approach can facilitate hot-swapping of models and/or retuning of parameters without interrupting overall data flow. In some implementations, the system 64 is configured to implement two-stage classification, wherein in a first stage, a “general exposure” classifier can be implemented to determine/detect an exposure or physiological state binary, which when positive, may trigger a subsequent higher-sensitivity process. At the subsequent stage, if the first stage determines a possible exposure or physiological state of interest, the second stage may determine/detect which agent or physiological state is specifically present/involved. Such multi-stage implementation can reduce false positives while maintaining prompt alerts for potential threats.

The AI classification engine 64 can comprise a random forest classifier model/engine 91 configured to implement an ensemble of decision trees 92. The system 64 may train N trees on bootstrapped subsets (e.g., random subsets) of the training feature data. Operationally, each tree 92 can be designed to split on features (e.g., baseline-subtracted, derivative analyte values) until a maximum depth or a minimum sample leaf size is reached. In real-time, each tree votes on a label (e.g., ‘mustard agent exposure” or “no exposure”). An ensemble/combiner module 81 can aggregate these votes (majority or weighted) to generate a final label 82. The random forest model 91 can include model loader circuitry 95, which may be configured to fetch the serialized tree ensemble from secure storage, verify its checksum and feature-vector compatibility, and/or de-serialize the file into an in-memory array of split-feature indices, thresholds, and/or leaf class probabilities. Once the forest is reconstructed (and optionally pruned for memory), the loader 95 may register a pointer to it so the inference thread can traverse the trees quickly and output real-time class votes or probability vectors. In some implementations, if an integrity check fails, the loader 95 may automatically roll-back to the last known good model to maintain continuous operation.

Each of the decision trees 92 may be implemented as a chain of conditional tests. Each node may encapsulate a concrete, real-time operation that turns the current feature-vector F (e.g., the engineered snapshot of analyte-related features, such as glucose, ketones, lactate, choline, oxygen, etc., and/or derivatives/ratios thereof) into a left-or-right routing decision. With respect to root tree nodes, such nodes may split on an analyte-based value (e.g., Δcholine), based on whether the relevant value is below or above a threshold/scalar value θ. During training, many candidate splits of the data may be evaluated (e.g., “is baseline-subtracted choline ≤−4 μM?” or “is lactate 1-min derivative >1 mM/min?”). For example, for each candidate split, the training samples may be partitioned into a left and right child node according to the threshold, wherein the Gini impurity of each child is determined and combined into a weighted impurity (e.g., weighted by how many samples fall in each child), the Gini impurity being a numerical measure of how “mixed” the class labels are in a set of training samples that reach the particular node in the decision tree. The split that minimizes the weighted Gini value may be chosen. Node thresholds may be picked in a manner as to yield the largest drop in Gini impurity (i.e., the cleanest separation of classes as early as possible), which can make the downstream path shorter and more accurate. The system 64 may maintain the node parameters (e.g., identifier(s) of the feature that splits the binary, the threshold/scalar value θ, etc.).

The root node of a tree may generally be the most discriminative binary determined during training (e.g., “has choline dropped by >4 μM?,” which can provide a relatively quick, early indicator for organophosphate exposure). For internal nodes, additional Boolean tests may be applied to split decisions to left and right child branches. Each internal node generally refines the upstream hypothesis. For example, in one use case, after determining that choline has dropped, the subsequent internal node may determine whether a lactate (or other analyte) 1st derivative is greater than a threshold (e.g., 1 mM/min), which may discriminate organophosphate toxins from opioid respiratory depression or other state of lesser concern. Internal node operation may involve generating and/or maintaining certain tree parameters, such as, for example, class counts for data that reached the respective node during training, and/or feature index masks (e.g., if certain features are pruned at deeper levels).

For leaf/terminal nodes, such nodes may encode one or more micro-rules determinative of a final classification (e.g., Δcholine≤−4 μM AND Δlactate≥2 mM AND ΔO₂<15 mmHg⇒organophosphate exposure with 82% confidence). The leaf/terminal nodes output the tree outputs, including the physiological/exposure state classification (e.g., ‘organophosphate exposure,’ ‘no exposure,’ ‘meal state,’ ‘exercise state,’ etc.), as well as a probability/confidence value associated with the classification. Leaf node operation may involve generating and/or maintaining certain tree parameters, such as, for example, a classification probability vector, indication of the majority label, training-sample count, feature mean/variance, etc.

The different ones of the trees 92 can test different feature subsets and thresholds, providing desirable robustness through diversity. For example, some paths may rely on glucose or oxygen dynamics, others on correlation scores, etc. Such tree diversity can advantageously mitigate over-fitting to any single artefact. Through chaining of the deterministic node operations, each tree can contribute an independent, interpretable vote toward the forest's overall real-time classification of physiological state and/or chemical exposure state (e.g., mustard, organophosphate, opioid, no exposure). The system 64 can be configured to determine which input signals are most influential to the various classifications, wherein such feedback can be stored in a trace log, enhancing real-time diagnostics and/or future model refinements. Due to the trees 92 being relatively shallow (e.g., max depth <8), real-time classification can be performed computationally efficiently, which is suitable for resource-constrained hardware (e.g., embedded microcontrollers of mobile computing devices).

The AI classification engine 64 can comprise gradient-boosting classifier model/engine 93 (e.g., XGBoost model or other gradient-boosted decision-tree ensemble) configured to implement gradient boosting on decision trees 94 to produce flexible classification. The gradient-boosting model 93 receives the pre-computed features/vector 58, which may comprise an ordered list of baseline-subtracted values, derivatives, ratios, and/or correlation metrics. The features 58 may be received by the gradient-boosting module 93 and/or other model(s) of the system 64 through a single input bus.

The gradient-boosting model 93 can include model loader circuitry 96, which can provide a mechanism to retrieve the most-recent parameters for the gradient-boosting model 93 from the persistence storage 61 into working memory. The model loader 96 can parse the binary trees 94 of the model, allocate additive-score arrays, and/or preload structural parameters (e.g., parameters representing maximum decision tree depth, fraction of features sampled per tree, fraction of features sampled per tree level, fraction of features sampled per node, fraction of training rows sampled per tree, learning rate, number of trees/boosting rounds, split-loss regularization, etc.).

The features 58 may be provided to the model loader 96, which pulls the most-recent gradient-boosted tree ensemble and/or its hyper-parameter data structure/file from the central model repository 61. Once loaded into memory, the features 58 can be provided to a booster chain 94, depicted as a horizontal series of tree icons (Tree 0 through Tree M). Each tree can perform a lightweight sequence of feature-to-threshold comparisons, routing a sample down to a leaf, and outputting a class-specific leaf score. Each new tree may correct residual errors of the previous ensemble. A score aggregator 97 can implement an additive model to continuously accrue the leaf scores for every class label.

The aggregated raw scores may be provided to logistic layer circuitry 98, where they are converted into a calibrated probability distribution 83 across the physiological state and/or exposure classes (e.g., meal state, sleep state, exercise state, mustard agent, organophosphate, opioid, etc.). These probabilities 83 are provided to the system's ensemble/model-selector circuitry 67, or in single-model deployments, directly to the output/alert-generation circuitry 87. An auxiliary data path may provide the probability distribution data structure 83 to a feature importance interpreter module 401, such as a SHAP (SHapley Additive exPlanations) module, which can be configured to generate output indicative of post-hoc interpretability of the probability distribution, indicating for specific classifications, how much each individual feature of the feature set 58 influenced the model toward or away from the final output classification. For example, the module 401 can include circuitry configured to compute per-feature contribution values, allowing the UI module 87 to log and/or display why the model 93 reached its conclusion.

The AI classification engine 64 can comprise a neural network classifier model/engine 71 configured to implement, for example, feed-forward layers for classification output generation, which may provide diversity to the system 64 as an alternative to the tree-based classifier(s). At start-up or when a new version is signaled, the neural network (NN) classifier 71 can retrieve the latest NN weights (e.g., floating-point weight tensors) and/or configuration file (e.g., indicating configuration such as layer order, activation functions, dropout rate, etc.) from model loader circuitry 73.

In some embodiments, the model 71 is a feed-forward neural network configured for real-time embedded inference/classification. The model 71 includes an input layer 76 that receives the feature set 58 and prepares the feature set data for processing. The input layer 76 may or may not transform the data, and may align each feature with a corresponding weight row of the hidden layer 72. The input layer may have as many nodes/neurons as there are input features in the feature set 58.

The feature set data is provided from the input layer 76 to the hidden layer 72, which may advantageously be implemented as a fully-connected dense layer between the input 76 and output 77 layers. Each hidden layer can comprise a plurality of artificial neurons (e.g., 64 units), wherein each neuron of a given layer receives a weighted connection from every feature value produced by the preceding layer. The hidden layer 72 may generate a linear transformation (e.g., activation vector) Z of the feature set data structure/vector, such as according to the equation Z=WF+b, where F is the feature data structure, W is a weight matrix and b is a bias vector, any of which may be maintained in the datastore 61. The activation/transformation Z may be passed through a non-linear activation function, such as a rectified-linear unit (ReLU), to obtain the hidden layer output, H. An example representation of the hidden layer output may be H=ReLU(Z). The activation function can enable the network to capture non-linear relationships among the multi-analyte features (e.g., simultaneous choline depression and lactate elevation characteristic of an organophosphate exposure). In some embodiments, a dropout regularization stage may be implemented to temporarily mask a fixed fraction (e.g., 25%) of the ReLU activations during training, compelling the model to rely on multiple, redundant feature combinations, thus improving robustness to sensor noise and inter-subject variability.

Operationally, the hidden layer 72 serves as a trainable feature-extraction stage by compressing the high-dimensional feature set into a lower-dimensional internal representation in which analyte patterns are amplified while irrelevant or highly-correlated inputs are attenuated. The hidden layer's output can be forwarded to the output layer 77, completing a low-latency feed-forward path that requires only a matrix-vector multiply, bias addition, and/or element-wise ReLU, which can be suitable for a resource-constrained wearable microcontroller while providing sufficient representational power to discriminate among physiological and/or chemical exposure states in real time. The probability distribution/vector 75 can be passed to the ensemble/model-selector module 67 or compared to per-class confidence thresholds to trigger an alert or other output. An optional integrated-gradients routine can be run, such as by the feature importance interpreter module 401, to generate and/or maintain per-feature attribution scores associated with the model output 75. Such scores may be surfaced to the user by the output module 87. Labeled data accumulated in the field can be periodically pushed to the NN training module 81 (see FIG. 3), which may reside locally or on a cloud server, at least in part.

The ensemble/model-selector 67 can transform the heterogeneous classifier outputs 82, 83, 75 into a single, threshold-qualified label 85 and/or confidence score/value 86. The ensemble/model-selector 67 may receive synchronous output data structures/packets from the AI classification models 91, 93, 71, which may include reporting model identifiers, probability vector lengths, hard labels, timestamps, and/or other data/metadata.

The ensemble/model-selector may be configured to implement model weighting, wherein a weight table or other mechanism may be utilized weight the model outputs, such as by multiplying the outputs by their respective weights. The outputs/probabilities of the models may be combined in a fusion operation through summing of the weighted outputs or other process, wherein the fused model output may comprise the final classification/label 85. A confidence value 86 associated with the classification 85 may be generated in any suitable or desirable manner, such as based on a margin between a most-likely class probability compared to a second-most-likely class probability associated with the probability distribution of the fusion output 85. For example, class-specific threshold(s) may be utilized (e.g., from a calibration table). In such implementations, for example, if the top probability is greater than the relevant class-specific threshold and/or the margin exceeds a maintained/predetermined margin threshold/value, the confidence 86 may be indicated as ‘strong,’ otherwise the confidence may be indicated as ‘weak’ or ‘uncertain.’ Alternatively, a gradient certainty may be determined based on the determined margin. In some implementations, the module 67 may implement a voting scheme, wherein agreement of classification label between two or more of the classification models results in a classification in agreement with such grouping. In accordance with the description above, the output of the ensemble module 67 may include the label/classification data structure 85, and may possibly include the confidence value/flag (e.g., indicating ‘strong,’ ‘weak,’ or ‘no exposure/state’) or numerical confidence score/value (e.g., value between 0-1).

The output(s) 85, 86 may be processed downstream by the UI/output module 87, which may be configured to cause a visual rendering of physiological state and/or chemical exposure state (e.g., color-coded, with red equating to strong exposure/condition, amber meaning weak, green meaning none, or similar). In some embodiments, probability bar-chart(s) may be generated showing the various class probabilities. When integrated-gradients or SHAP data are available, the output may include indications of the top contributing features.

The UI/output module 87 can be configured to implement an alert policy. As an example use case, related variations of which are within the scope of the relevant disclosure, a detected strong state/exposure may trigger an immediate push notification to the user, audible tone, and/or vibration. The module 87 may further log an entry in the datastore 403 marking the event and its associated severity/strength (e.g., ‘HIGH’). For weak strength/exposure, a passive banner output, no tone, or similar may be used, whereas for no detected state/exposure background logging may be implemented without any user output. For strong events, the module 87 may trigger a notification to a remote command/control server. The UI/alert module 87 may be configured to convert the classification 85 into human-actionable information, including visual status, audible/haptic signals, and/or, when necessary, automatically escalated incident reports. The output module 87 may wait for user confirmation (“Acknowledge” button) or a configurable timeout before initiating certain responsive action.

### Classification Model Training

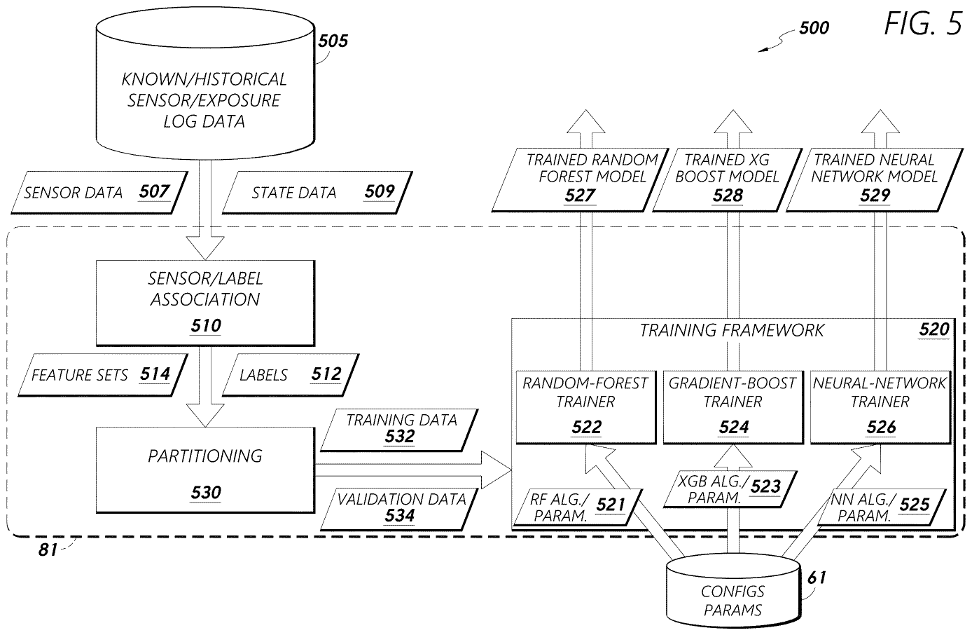

FIG. 5 is a data transformation/flow diagram illustrating systems and processes for training artificial intelligence (AI) model(s) for physiological condition/state classification in accordance with one or more embodiments. In particular, FIG. 5 shows a flexible, data-driven model-training architecture 500 that converts archival sensor logs into production-grade classifiers suitable for real-time deployment. The architecture 500 can utilize known/historical sensor-exposure log data from a data repository 505. Certain sensor data 507 and corresponding known physiological state data (e.g., exposure data) 509 indicating states associated with the sensor data 507 can be utilized by the architecture 500 as a basis for AI model training. For example, the sensor data 507 can include data/signals from various analyte and/or physical sensors, such as sensor data indicating levels/signals relating to lactate, choline, oxygen, glucose, ketones, reactive oxygen species (ROS), temperature, motion, and/or the like. The state data 509 can provide ground-truth annotations that mark physiological or toxicologic episodes (e.g., ‘mustard exposure,’ ‘opioid dose,’ ‘rest,’ ‘exercise,’ etc.) and/or associated metadata (e.g., start and stop times, confidence scores, etc.).

The training system/circuitry 81 of the architecture 500 can access the synchronized feeds 507, 509 and use such data for model training. For example, the sensor data stream 507 can provide raw numerical vectors in chronological order, while the state data stream 509 can supply the associated episode records. The data feeds 507, 509 can be delivered to sensor/label-association circuitry 510, which may perform certain transformations on the data to generate feature set 514 and label 512 data associated with the sensor data 507. The association circuitry 510 can perform signal conditioning and feature engineering functionality, wherein filtering, baseline subtraction, derivative calculation, rolling-window statistics, pair-wise ratio, and/or inter-sensor correlation transformations may be implemented to produce a dense engineered vector for each analysis window. For example, the association circuitry 510 may perform similar functions as the transformation circuitry 50 shown in FIG. 3 and described above to generate features that correspond to the feature set used in real-time classification, such that the training framework 520 can have correspondence to the real-time classification framework 64. The association circuitry 510 may further perform quality filtering to cull data captured during probe run-in or periods of diagnostic failure so that drift artefacts do not contaminate the learning set. The association circuitry 510 may further perform certain temporal alignment and/or label propagation to locate, for each vector, an interval record having time bounds that enclose the vector's timestamp (if any), and further to assign the corresponding class label. The association circuitry 510 can further perform encoding and manifesting functionality, wherein feature order, scaling constants, label encodings, and/or dataset integrity hashes/metadata are generated and/or maintained to promote correct interpretation by downstream functions.

The association module/circuitry 510 generates and/or provides, for each slice of data (e.g., timestamp), a feature-set matrix 514 and a label vector 512, which together represent an analyte-by-time classification correspondence. Such data may flow to partitioning circuitry 530, which can be configured to create the final training subsets for learning and evaluation. The partitioning circuitry 530 can implement any suitable or desirable partitioning of the data, such as data hold-out, k-fold cross-validation, and/or leave-one-subject-out partitioning, which may be dictated by a selectable policy profile. The partitioning circuitry 530 can implement stratification routines to maintain class proportions across splits, wherein subject-or session-isolation rules can ensure that all data from a given individual or test run reside wholly in either the training subset 532 or the validation subset 534 generated/provided by the partitioning module 530. Temporal blocking can further be implemented to prevent adjacent windows from straddling the split. Optional imbalance controls, such as random under-sampling, synthetic oversampling, or class-weight assignment, can be implemented to improve results. The partitioning module 530 can be configured to record row indices, random seeds, and/or applied options in a split manifest to allow for splits to be reconstructed.

As shown and referenced, the partitions 532, 534 can be routed to a training framework 520, which can advantageously be configured to perform model fitting across multiple algorithm families. Internally, the training framework 520 can host one or more of three learner blocks: a random-forest trainer 522, a gradient-boost (e.g., XGBoost) trainer 524, and a neural-network trainer 526. Each trainer can request/access a hyper-parameter profile from a central configurations/parameters datastore 61, illustrated as random forest parameters 521, gradient-boost parameters 523, and neutral-network parameters 525. For example, tree depth limits for the forest, learning-rate schedules for boosting, and/or layer sizes and dropout rates for the neural network can be maintained in the datastore 61 and provided to a shared bus.

The random-forest trainer 522 can be configured to build an ensemble of decision trees on the training subset 532, optionally monitoring Gini or entropy reduction and pruning criteria. For example, the accumulating forest can be periodically scored on the validation subset 534 to detect over-fitting and determine an optimal tree count. The gradient-boost trainer 524 can be configured to construct an additive sequence of shallow trees, each fitted to the residual error of the previous ensemble. Shrinkage, feature-sampling, and/or early-stop logic can be applied according to the parameter profile. The neural-network (NN) trainer 526 can be configured to instantiate the prescribed topology, standardize feature inputs, perform batch-optimized weight updates, and/or halt when validation loss fails to improve for a predefined patience window.

Each trainer may generate a model artefact (e.g., trained random-forest model 527, trained gradient-boost model 528, trained neural-network model 529), which may be provided together with auxiliary data, such as feature-importance rankings, calibration curves, and/or confusion matrices. The training framework 520 may run the trainers sequentially or in parallel, depending on available compute resources. The training framework 520 may further tag each artefact with a version identifier and/or store the accompanying metrics so that downstream deployment logic can select the most suitable model or form an ensemble. The combined functions of the coordinated data-provisioning 505, association 510, partitioning 530, and multi-algorithm training 520 modules can allow the architecture 500 to generate rigorously validated classifiers that are ready for packaging and upload to the real-time inference/classification subsystem 64 of the multi-analyte monitoring platform/system 10.

### User Interface

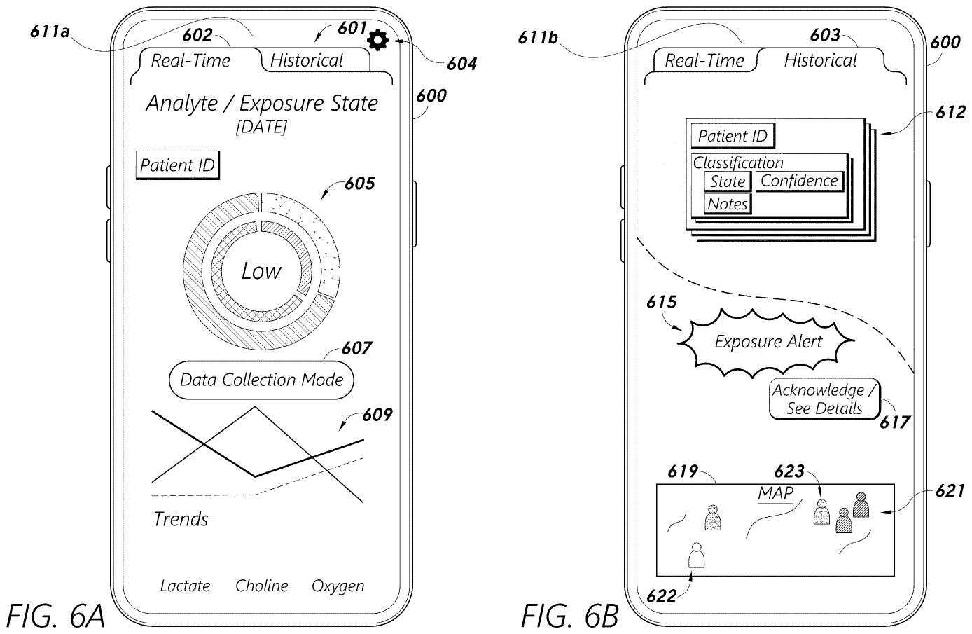

FIGS. 6A and 6B show example user interface elements for a multi-analyte state determination and monitoring system in accordance with aspects of the present disclosure. The interfaces 611a, 611b of FIGS. 6A and 6B are shown on a smartphone display/application, though it should be understood that such features can be implemented on any mobile or non-mobile computing device. The illustrated mobile application(s) can be installed on a user's personal device 600 or on a dedicated field device carried by a soldier, first responder, or medical personnel, for example. In some embodiments, the mobile device 600 connects to a remote server for cloud-based data analysis or centralized command monitoring, as described in detail herein.

A short-range or mid-range protocol (e.g., Bluetooth, NFC, Wi-Fi) can be used by the device 600 to receive sensor data from a wearable multi-analyte sensor implant. The interfaces 611a, 611b can include graphical elements, which may be represented by graphical interface data generated by any component of the system 10 of FIG. 1, showing various features relating to analyte and/or physiological state conditions. For example, the interface 611a shows current analyte readings (e.g., lactate, oxygen, choline), which may be represented as line graphs/plots 609, bar graphs, or other infographics (e.g., circle graph 605), and/or as numeric values. In some implementations, analyte readings can be accompanied by a trend icon (up arrow for increasing, down arrow for decreasing) based on short-term rolling statistics.

Interface 611b shows an example physiological state detection alert icon 615, which may be displayed if the AI model classifies an event, such as a chemical exposure or noteworthy physiological state change (e.g., meal intake). The alert 615 may change color (e.g., red for a confirmed threat, yellow for uncertain) and may produce a vibration or audible alarm on the device 600. A corresponding button 617 may be implemented for acknowledging the alert and/or triggering/requesting presentation of additional details, such as recommended protective measures or emergency instructions. The interface 611b further shows functionality for switching to a “Historical,” or “Logs,” screen/interface 603 that may provide previous sensor readings, classification events, and any user annotations. Such logs 612 may be chronologically listed and/or searchable by keyword and/or filtering for convenience. Logs 612 may show physiological state classifications, date/timestamps, classification confidence levels, and/or user-entered notes.

A scrollable or tabbed interface 601 may be implemented to allow easy navigation between different analytes or different time windows (e.g., last 24 hours, last 7 days). An icon 604 or menu may further be included that opens to reveal sensor calibration options, threshold preferences, alert volume or vibration settings, and/or network connectivity settings. The user may be able to toggle advanced features, such as whether to upload anonymized sensor data to a cloud repository for model retraining or enable remote monitoring by command center staff. In some implementations, the interface 611a includes a data collection mode initiation button 607, which may trigger sensor readings and/or state classification.

In some implementations, a multiple-subject (e.g., soldier) avatar map 619 (e.g., geographic information systems (GIS) map) may be implemented, wherein locations of subjects in one or more regions are represented at positions on the map 619. The avatars displayed on the map 619 may have status icons or features indicating physiological state/exposure conditions. For example, the illustrated map 619 shows a first subset of subjects 621 with a first color, fill, or other indicator that indicates a chemical exposure or relatively serious physiological state. Other subsets 623, 622 of subject avatars may be otherwise colored/filled to indicate other state conditions. The device interface can thereby centralize data from a squad or group, enabling a medic or commander to track group safety. Within the monitoring system, physiological state/exposure information from multiple subjects and/or multiple groups of subjects may be used to identify geographically safe and/or hazardous areas of the map 619.

The diagrams of FIGS. 6A and 6B demonstrate how the specialized multi-analyte sensing and physiological state classification systems disclosed herein can be translated into an actionable user interface, which goes beyond mere abstract data processing by integrating physical sensors (e.g., subdermal/wearable), real-time AI classification, and user-facing smartphone applications in a manner that transforms raw signals into clear alerts and historical logs, addressing a technical problem of timely and accurate physiological state and/or chemical threat notification.

### Multi-Analyte Profiles for State/Exposure Classification

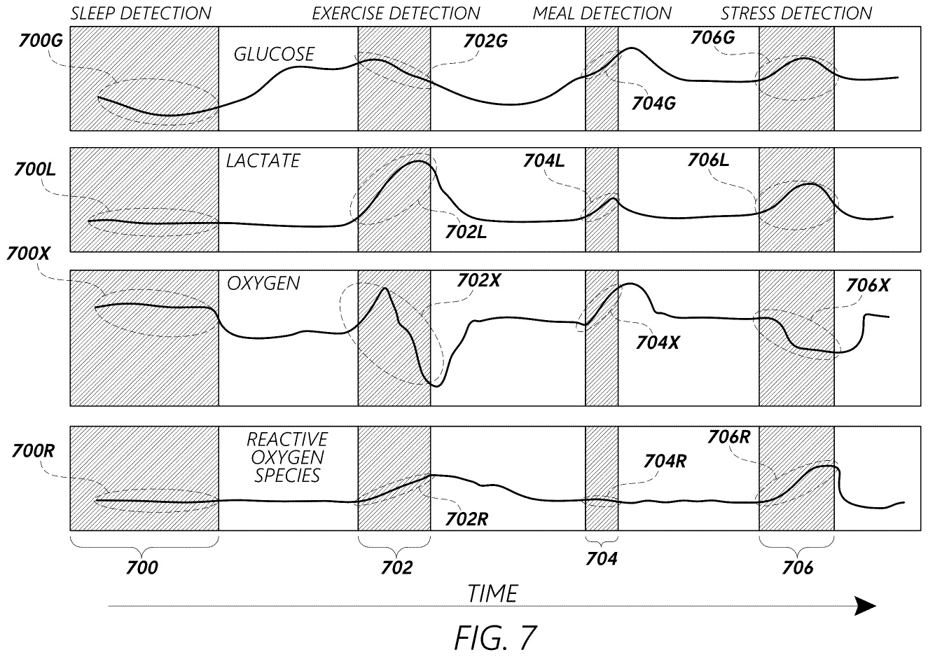

The graph set presented in FIGS. 7-10B visually illustrates the characteristic analyte response patterns that can underlie the classification logic of physiological state classification engines and systems disclosed herein. Each plot overlays time-aligned/time-series traces of measured analytes with the ground-truth physiological or exposure labels, identified by vertical bands in the graphs, acquired during controlled trials. The graphs demonstrate distinct signature envelopes that systems of the present disclosure are trained to identify as indicating respective physiological states/classifications. The illustrated signal combinations represent non-limiting examples of multi-analyte signal relationships that can form the data foundation on which the random-forest, gradient-boost, and/or neural-network models of the present disclosure are trained, and the graphs therefore provide an intuitive, empirical backdrop for the AI-derived decision boundaries described in this disclosure. In FIGS. 7-10B, the durations of the various physiological states are not intended to be to scale. Rather the physiological states and the exemplary data/curves within the duration ranges of the physiological conditions are intended to illustrate exemplary trends and rates of change. In practice, it may be necessary to calibrate detection algorithms using days, weeks, or even months of individual patient data. Note that though the detected physiological states are shown as independent episodes, physiological states are not necessarily exclusive in time and it is possible that multiple physiological states may be detected simultaneously.

FIG. 7 is an exemplary illustration of real-time glucose, lactate, oxygen, and ROS data associated with various physiological states such as sleep 700, exercise 702, a meal 704 and stress 706, in accordance with aspects of the present invention. In FIG. 7, sleep glucose values 700G and sleep lactate 700L are actively being acquired starting from the left to the right. Initially 700G is in a shallow decline that eventually levels out with even a slight increase toward the end of the sleep state. Similarly, sleep lactate 700L is illustrated as a relatively flat line. Accordingly, in some embodiments prolonged periods of steady lactate data in conjunction with decreasing to leveling glucose data can result in an initial detection of a sleep state. In embodiments where analyte sensors are supplemented by physical sensors, exemplary physical data acquired by accelerometers, thermometers, elapsed time, and ECG systems can confirm or refute the detection of a sleep state by the analyte sensor data. In preferred embodiments, analyte sensor data and physical sensor data are examined simultaneously for an initial detection of a sleep state and continued analysis of analyte and sensor data is used to confirm or refute the initial detection.

Real-time glucose and lactate data can be utilized by systems of the present disclosure to automatically detect/determine an exercise state 702. Exercise glucose 702G and exercise lactate 702L are exemplary and are not intended to be indicative of any particular individual or group of individuals. What is to be noted for each analyte is the combination of absolute change and rate of change. As exercise is performed, glucose levels may initially rise and then generally decrease. Similarly, during aerobic exercise, lactate levels may gradually increase until the exertion becomes anaerobic, where lactate levels may increase at a more rapid rate.

Meal consumption can also be identified based on real-time glucose and lactate data. Meal glucose 704G and meal lactate 704L each show increases during, or shortly after, a meal. It may be beneficial to supplement analyte sensor data with time when attempting to detect a meal state. Specifically, it may be very beneficial to include historical mealtimes within an algorithm to assist in distinguishing between a meal state and an exercise state. While the meal glucose 704G data and meal lactate 704L data is exemplary, rates of change and absolute value changes of each analyte across various meals and roughly the same time can help detect a meal state with increasing confidence.

Stress is another physiological state that may be detected/determined by systems of the present disclosure, using a combination of analyte and physical sensors. Stress glucose 706G and stress lactate 706L are exemplary illustrations of data obtained during a period of stress. In the exemplary data both glucose and lactate increase and decrease a relatively modest amount. Again, the exemplary data can be supplemented by physical sensor data that can be used to increase confidence in detecting stress.

FIG. 7 includes exemplary illustrations of sleep oxygen 700X, exercise oxygen 702X, meal oxygen 704X and stress oxygen 706X, which can be used in conjunction with glucose or other analytes to help detect the various physiological states. Oxygen changes in response to exercise and meals and can enable more reliable detection of sleep and/or quality of sleep than accelerometers. In some implementations, as accelerometers can be included as physical sensors, sleep oxygen 700X and be detected via either analyte or physical sensors and further confirmed using either or both analyte or physical sensors.

FIG. 7 further includes exemplary illustrations of sleep ROS 700R, exercise ROS 702R, meal ROS 704R and stress ROS 706R. Concentration of ROS in the body provides insight into oxidative stress. Additionally, oxidative stress can impact the timing of postprandial glucose excursions, causing a delay in glucose decrease after a meal. Because of the involvement of oxidative stress in many diabetes related complications, concentration of ROS can also provide information on the risk of developing secondary diseases.

With regard to ketone signals (not shown in FIG. 7), prolonged fasting can be signaled by progressive rise in β-hydroxybutyrate as hepatic fat oxidation increases. Exercise-induced metabolic stress (especially endurance activity) can be signaled by moderate, time-dependent elevation in ketones as glycogen stores are depleted. Post-meal recovery state can be associated with suppression of ketones after carbohydrate ingestion, re-elevation as insulin falls. An insulin-deficient state (e.g., incipient ketoacidosis) can be signaled by rapid and pronounced rise in ketones accompanying hyperglycaemia. A sleep state classification can be triggered by gradual nocturnal increase in ketones, followed by decline with breakfast.

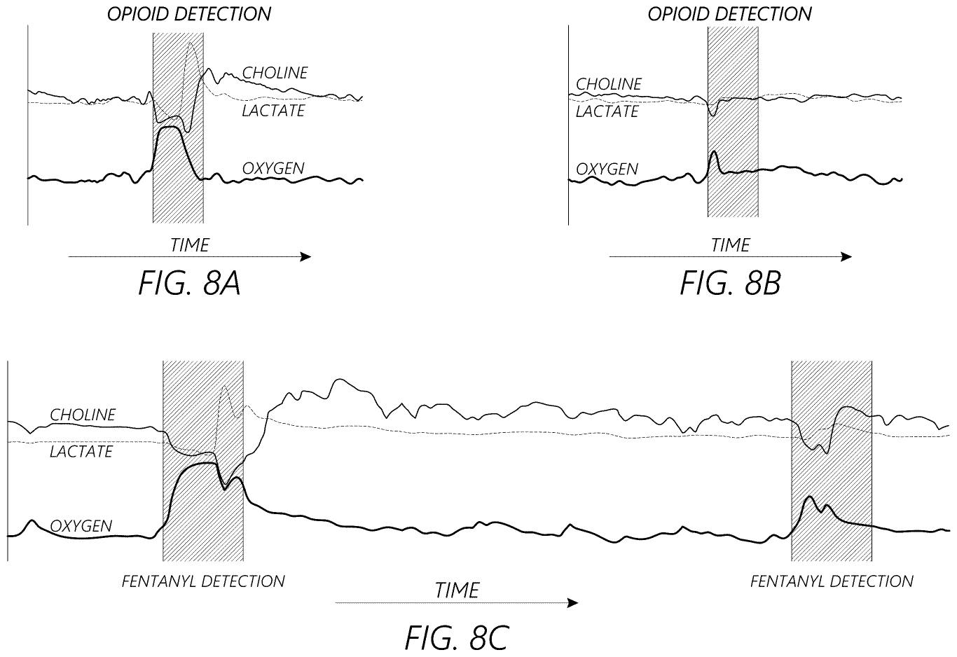

FIGS. 8A, 8B, and 8C illustrate an example multi-analyte signature that classification engines as described herein can identify as a basis for acute opioid exposure detection. As shown, opioid exposure can be determined through AI model recognition of a choline sensor signal exhibiting a rapid, monotonic decline, reflecting reduced acetylcholine turnover that is characteristic of μ-opioid-mediated parasympathetic suppression. Concurrently, or almost concurrently, the oxygen signal may show a transient upward inflection, which represents a response consistent with transient hypoventilation followed by compensatory hyperoxia as respiratory drive is pharmacologically modulated by the opioid exposure. The lactate trace may taper modestly during this hypoxic interval, then rebounds and climbs as compensatory anaerobic metabolism ensues. The coincident timing of a steep choline drop, an oxygen dip, and/or a biphasic lactate response can form a reproducible pattern that the trained models of the present disclosure are trained to recognize. This tri-sensor trajectory lies in a distinct region that the random-forest, gradient-boost, and neural-network classifiers collectively or individually may map to the ‘opioid exposure’ classification with relatively high probability and/or confidence.

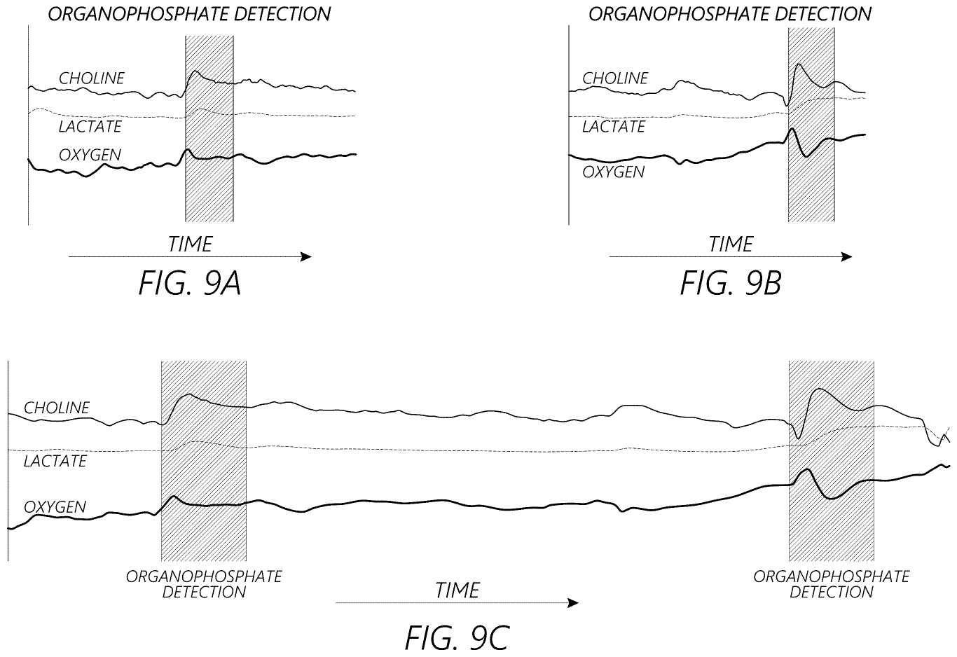

FIGS. 9A-9D illustrate example multi-analyte patterns that classification engines as described herein can identify as a basis for organophosphate (OP) exposure detection. As shown, an early, pulse-like increase in the oxygen channel may be observed first. Such brief hyperoxic surge is consistent with an immediate, cholinergically driven spike in respiratory effort following acetylcholinesterase inhibition. The choline signal may further begin a sharp ascent as excess acetylcholine is hydrolyzed to choline that the sensor registers. Both choline and oxygen subsequently may decline toward baseline once compensatory mechanisms engage. The lactate trace may lag the other two, displaying a modest rise that peaks later (e.g., several minutes later), reflecting a shift toward anaerobic metabolism as sustained cholinergic stress impairs efficient oxygen utilization, before tapering off. Analyte signal profiles as shown in FIGS. 9A-9D can trigger AI systems as disclosed herein to make a classification/determination of organophosphate exposure. For example, with respect to operation of AI models as presented herein, the illustrated and described temporal ordering (e.g., oxygen rise leading choline rise, followed by a delayed, lower-amplitude lactate elevation, with all three signals returning toward baseline) can cause the AI model(s) to generate feature values that consistently fall near one another and away from points generated by other conditions (e.g., opioid or mustard exposure). That is, such signal patterns can form tight clusters (referred to as a “motif”) occupying its own corner of the feature space, such that the classifier learns to treat any point landing in that region as evidence of organophosphate exposure. The trained random-forest, gradient-boost, and/or neural-network models reliably map this motif to the ‘organophosphate exposure’ state/class with high confidence.

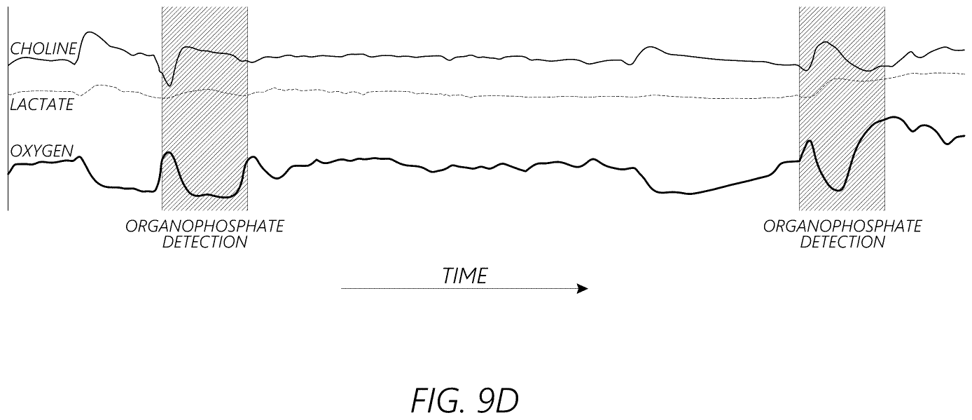

FIGS. 10A and 10B depict representative multi-analyte signal profiles that the disclosed classification engines can associate with mustard-agent exposure. In the example illustrated traces, the oxygen signal/channel exhibits the dominant excursion, namely a pronounced, steep rise that peaks rapidly and then decays toward baseline, consistent with an acute hyperventilatory response to early tissue irritation followed by compensatory normalization. The choline signal follows (e.g., a few seconds later) with a more modest, broad-topped increase, which may reflect delayed cholinergic activation secondary to cellular injury, and likewise returns gradually to its pre-event level. The lactate channel shows only minor movement, typically a shallow rise and fall that remains within a narrow band, indicating that systemic anaerobic demand is present but limited in magnitude and duration. This temporal ordering (e.g., oxygen surge leading a smaller, lagged choline elevation, accompanied by minimal lactate displacement) can cause AI model(s) as disclosed herein to generate a constellation of feature values that occupies a region of feature space distinct from those produced by organophosphate or opioid events. During model training, the random-forest, gradient-boost, and neural-network learners can capture this mustard motif as a tight cluster. During real-time classification operation, any newly computed feature vector that lands in or near that cluster is classified as mustard-agent exposure (e.g., with high confidence), thereby triggering the appropriate alert or downstream response.

In some implementations, glucose, lactate, and choline sensor signals/readings serve as the basis for physiological state classification. The combination of these three analytes in connection with systems disclosed herein can be advantageous as providing three, partly orthogonal, biological axes (namely energy availability, aerobic-to-anaerobic metabolic balance, and cholinergic/autonomic tone) that, when analyzed together, can allow disclosed inventive classifiers to resolve a certain physiological and toxicological conditions that may remain ambiguous if any single channel of the three were absent. For example, a rapid post-prandial rise in glucose accompanied by only a gentle lactate drift and essentially baseline choline can be interpreted by systems disclosed herein as an absorptive (meal) state, whereas a pronounced, sustained lactate surge coupled with falling or flat glucose and a modest choline elevation is characteristic of high-intensity, largely anaerobic exercise. Overnight sleep or prolonged fasting can present as a gradual glucose decline with lactate and choline remaining quiescent, while acute psychological stress may be marked by a catecholamine-driven glucose spike together with parallel, moderate lactate elevation and a small, transient choline excursion. Distinct toxicological patterns can likewise be detected by disclosed classifiers based on analysis of these analytes. For example, organophosphate exposure can be detected in response to an early hyper-cholinergic peak followed by a secondary lactate rise with little change in glucose, whereas opioid overdose may be detected as an abrupt choline suppression, minor biphasic lactate elevation, and negligible glucose perturbation. Because these combinatorial signatures occupy well-separated regions in the engineered feature space (e.g., derived from baseline-subtracted values, first-order derivatives, and/or pairwise ratios), random-forest, gradient-boost, and neural-network models described herein can assign high-confidence labels such as “meal,” “exercise,” “sleep,” “organophosphate exposure,” or “opioid exposure” in real time, enabling the disclosed systems to initiate context-appropriate alerts, therapy adjustments, or remote notifications with minimal latency.

### Multi-Analyte Sensor Probe Layouts

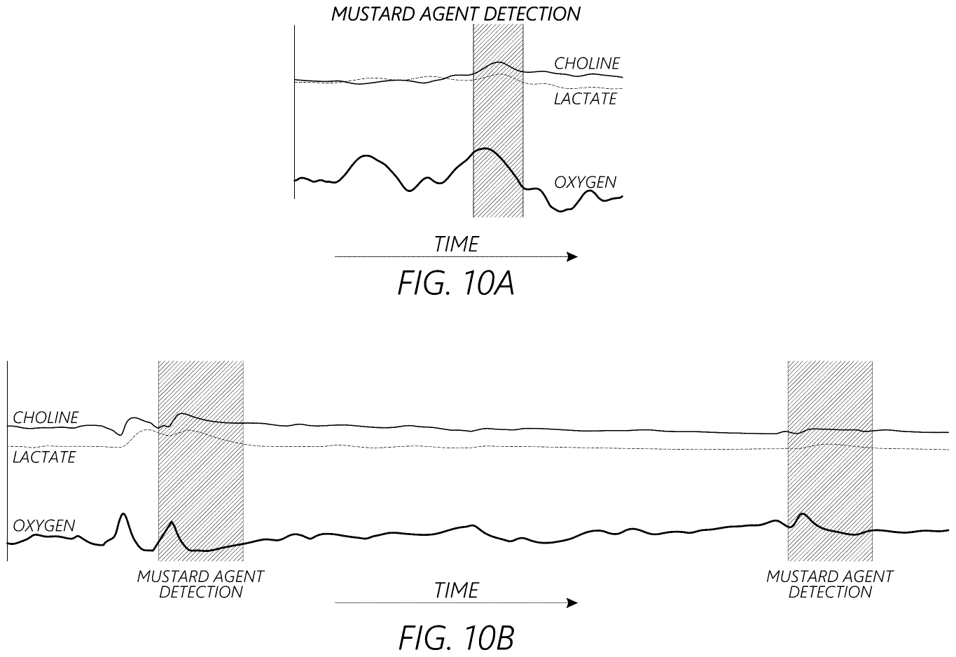

To supply the AI-based classification engine with the high-fidelity, multi-analyte signals that serve as the basis for physiological state classification, the system physical sensor probe(s) can advantageously be configured with working, reference, and counter electrodes positioned/oriented in a geometry that maximizes analyte specificity while suppressing electrochemical cross-talk. FIGS. 11A-13B illustrates representative probe layouts, showing the placement of working electrodes for glucose, lactate, ketone, choline, oxygen, and/or ROS sensing, two or more of which may be positioned on opposing faces of a biocompatible substrate. Such figures further demonstrate example routing of shared reference and counter electrodes, diffusion-limiting membranes, and/or insulating trenches. These structural arrangements provide example hardware foundations that can advantageously enable the multi-analyte sensor probe(s) to stream simultaneous, low-interference measurements of multiple biomarkers, which in turn constitute the feature vectors ingested and classified by the machine-learning models described throughout the present disclosure.

FIGS. 11A and 11B depict a dual-sided sensor array that is configured to provide multi-analyte data streams for utilization of AI-based classification engines as described herein. The array's elongate substrate defines an A-side 800a (FIG. 11A) and a B-side 800b (FIG. 11B) bounded by distal 801a and proximal 801n ends. On the A-side, a first conductor trace 804 terminates in multiple first electrode openings 802 that expose a first working electrode dedicated to a first analyte, such as glucose. Each opening 802 may be over-coated with first transport material 838 and first reactive chemistry 840. A second trace 808 carries second electrode openings 806 to a second working electrode functionalized by second reactive chemistry for a second contextual analyte (e.g., lactate, tissue oxygen, choline, β-hydroxybutyrate, or ROS). A third trace 812 supports third electrode openings 410 to a third working electrode that can duplicate the second analyte or target a third biomarker; this pad can be covered by a fourth transport material when configured as a counter/reference.

In some implementations, the B-side 800b can host the return circuitry. For example, traces 816 and 820 can end in combined counter/reference electrodes 814, 818, allowing two-electrode operation for all A-side working pads. A dedicated trace 824 can carry a third counter/reference electrode 822 to provide an independent reference for drift-sensitive channels or to perform electrochemical impedance spectroscopy (EIS) for tissue-hydration context. One or more additional working electrode traces may be implemented on the A or B side(s). Spatial ordering can advantageously minimize cross-talk. For example, oxygen and choline sites can be offset laterally from glucose sites, whereas lactate or ketone sites reside near distal tip 801a so that all metabolic markers sample the same micro-environment. Transport layers (e.g., 842 in FIG. 11C) and insulating trenches can be implemented to isolate diffusion domains so that peroxide from glucose oxidation, for example, does not reach the oxygen pad. All electrodes may share a single insertion pathway, enabling percutaneous deployment into subcutaneous tissue with minimal discomfort while furnishing tissue-level rather than arterial analyte dynamics. Any electrode pair can be repurposed transiently for EIS scans, giving the AI engine continuous fluid-status context.

Although FIGS. 11A and 11B show a two-face, single-shank layout, the disclosure encompasses mirrored or multi-shank variants that preserve at least one glucose working electrode, one or more auxiliary-analyte working electrodes, and/or suitable counter/reference electrodes. Such structural elements can advantageously provide the hardware foundation that generates the feature vectors classified by the random-forest, gradient-boost, and neural-network models disclosed in detail herein.

FIG. 11C schematically shows the micro-diffusion pathway used by each glucose channel in the example sensor array of FIG. 11A, whose stable output can serve as one of the key inputs to the AI-based physiological-state classifier. FIG. 11C is described in the context of glucose sensing, though it should be understood that aspects of the configuration the structure shown in FIG. 11C can be used for other types of analyte sensors. With respect to the illustrated example, glucose from interstitial fluid diffuses through a hydrophilic first transport layer 838, encounters a thin film of glucose-oxidase chemistry 840, and is oxidized according to equation (1) below:

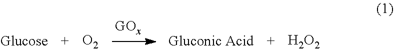

The hydrogen peroxide migrates laterally and vertically through layer 838 to the working-electrode body 836, whose reactive surface 816 is exposed by the insulator window 832. There the peroxide is electro-reduced according to equation (2) below:

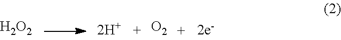

The resulting current is collected against a counter electrode. Because the enzyme zone 840 is physically separated from the electrode by the diffusion layer 838 and backed by a hydrophobic second transport layer 842, local pH shifts are minimized, cross-talk with adjacent analyte channels is suppressed, and manufacturability is simplified (like material on like material). The arrangement also can establish a self-refreshing diffusion gradient that keeps glucose influx stable, providing the classifier with a low-noise glucose feature that can be fused with simultaneous lactate, oxygen, choline, or ketone streams to identify sleep, exercise, meal intake, or chemical-exposure states in real time.

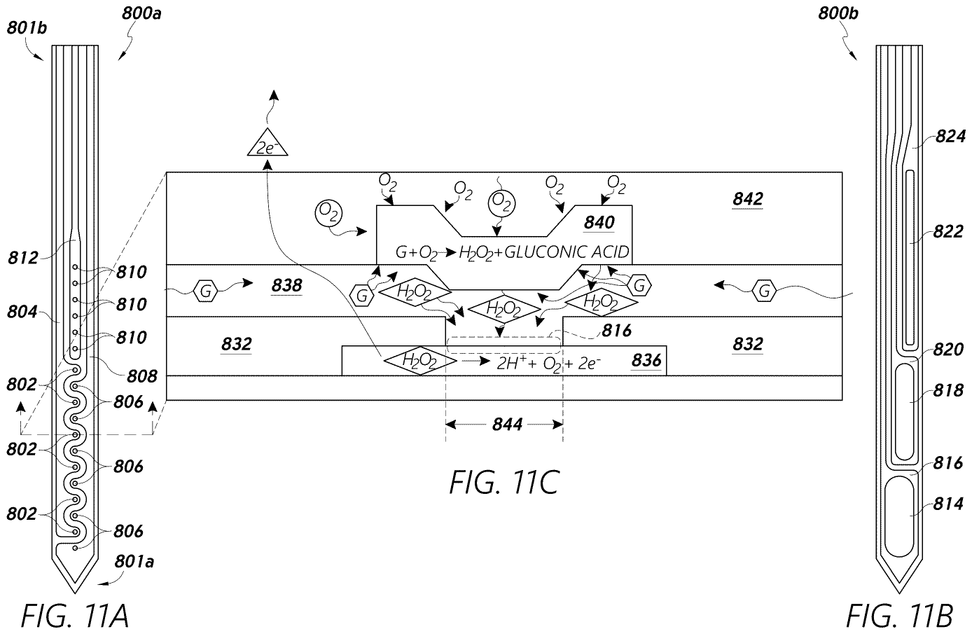

FIG. 12A shows an A-side 990a configuration of a dual-counter/reference sensor probe layout, in which the third electrode trace 812 carries a third counter/reference electrode opening 812a and overlying fourth transport material 906, thereby placing a dedicated pseudo-reference pad on the same face that already hosts the first working electrodes 802 and their first reactive chemistry 840 as well as the second working electrodes 806. FIG. 12B depicts the corresponding B-side 990b, where the fourth trace 900 terminates in a fourth counter/reference electrode opening 900a coated by fifth transport material 908. By distributing counter/reference sites on opposite faces, one near the distal end 901a, one near the proximal end 901b. Such layout can allow the on-board potentiostat array to dedicate an isolated reference to each analyte channel or to run electrochemical impedance spectroscopy between orthogonal trace pairs, improving signal stability in highly perfused or oedematous tissue and supplying the AI classifier with cleaner feature inputs.

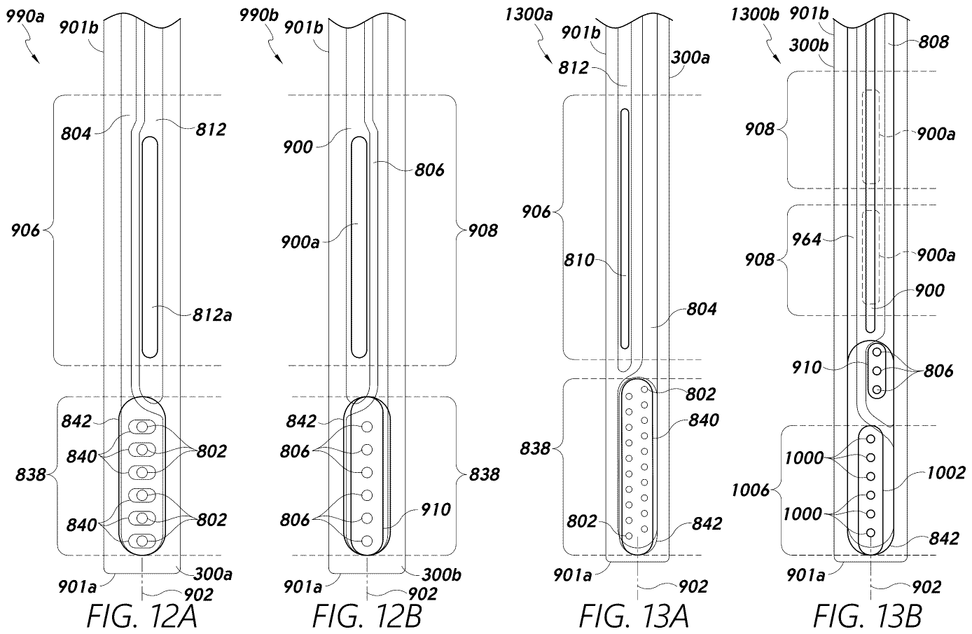

FIGS. 13A and 13B show configurations of an example three-analyte, mixed-reference sensor probe layout. In FIG. 13A, the A-side 1300a carries two distinct functional lines, namely a first trace 804 with multiple first openings 802, first transport material 438, and first reactive chemistry 840 (e.g., for glucose) and a third trace 812 with third opening 810 and fourth transport material 906, which may be repurposed as a first counter/reference electrode situated closer to the proximal end 904b.

FIG. 13B presents the complementary B-side 1300 b. A second trace 808 bears second openings 806 and second reactive chemistry 910 for a first auxiliary analyte (e.g., lactate or oxygen). Down the center-line 902, a fourth trace 900 and fourth opening 900a can supply a second counter/reference electrode, while a fifth trace 1004 with fifth openings 1000 supports third working electrodes coated with third reactive chemistry 1002 for a second auxiliary analyte (e.g., ROS or ketone). The entire B-side working region can be over-coated with second transport material 842, and the counter/reference pads with fifth transport material 908. This three-analyte layout can advantageously enable physiological state classification systems as disclosed herein to stream a first analyte (e.g., glucose) as well as two additional context biomarkers, which may be useful for distinguishing sleep, exercise, and meal states, while allocating independent reference potentials to drift-sensitive channels and leaving the remaining pads available for two-electrode operation or periodic bio-impedance scans.

Together with the dual-sided baseline design of FIGS. 11A and 11B, the FIG. 12A/12B and FIG. 13A/13B variants illustrate how different electrode-placement strategies (e.g., single shared pseudo-reference, dual distributed pseudo-references, or mixed dedicated references) can be chosen to balance miniaturization against signal isolation. The various illustrated configurations can preserve a single insertion pathway and essential structural features (e.g., working electrodes 802, 806, 810; counter/reference electrodes 814, 818, 822, 900a; transport layers 838, 842, 906, 908) that feed the multi-analyte AI classification pipelines described throughout the present disclosure.

### Additional Description of Examples

Provided below is a list of examples, each of which may include aspects of any of the other examples disclosed herein. Furthermore, aspects of any example described above may be implemented in any of the numbered examples provided below.

Example 1: A physiological state monitoring system comprising an implantable multi-analyte sensor probe including a first working electrode configured to generate a first electrical signal proportional to an in-tissue concentration of a first analyte, and at least one second working electrode electrically isolated from the first working electrode and configured to generate a second electrical signal proportional to a concentration of a second analyte. The system further comprises an electronics module electrically coupled to the sensor probe and including analog front-end circuitry configured to convert the first and second electrical signals into time-stamped digital sensor data, and a wireless communication interface. The system further comprises monitor system control circuitry communicatively coupled to the wireless communication interface and configured to receive the digital sensor data from the electronics module over the wireless communication interface, generate a feature vector that includes one or more of a baseline-subtracted analyte value, a first-order temporal derivative, a second-order temporal derivative, or an inter-analyte ratio based on the digital sensor data, access parameters of a machine-learning classification model trained on historical multi-analyte sensor logs and associated ground-truth physiological states and use the machine-learning classification model to generate, based on the feature vector, a real-time classification label and an associated confidence value identifying a physiological state.

Example 2: The physiological state monitoring system of any example presented herein, in particular example 1, wherein the machine-learning classification model comprises an ensemble including at least two of a random-forest classifier, a gradient-boosted decision-tree classifier, or a feed-forward neural-network classifier.

Example 3: The physiological state monitoring system of any example presented herein, in particular example 1, wherein the monitor system control circuitry is further configured to apply filtering and smoothing to the digital sensor data to remove high-frequency noise and electrode run-in drift prior to generating the feature vector.

Example 4: The physiological state monitoring system of example 1, wherein the physiological state is selected from a group of physiological states consisting of a plurality of: sleep, exercise, stress, meal ingestion, mustard-agent exposure, organophosphate exposure, opioid exposure, and no exposure.

Example 5: The physiological state monitoring system of any example presented herein, in particular example 1, wherein the multi-analyte sensor probe includes a first set of electrode windows exposing the first working electrode, and a second set of electrode windows exposing the second working electrode, wherein the first and second sets of electrode windows are interleaved along a central line of the multi-analyte sensor probe.

Example 6: The physiological state monitoring system of any example presented herein, in particular example 5, wherein the multi-analyte sensor probe further includes a third set of electrode windows exposing a third working electrode, the third set of electrode windows being positioned proximal of the first and second sets of electrode windows.

Example 7: The physiological state monitoring system of any example presented herein, in particular example 1, wherein the monitor system control circuitry is further configured to transmit the real-time classification label and the confidence value to at least one of a local user interface or a remote monitoring server.

Example 8: The physiological state monitoring system of any example presented herein, in particular example 1, wherein the first and second analytes are selected from the group consisting of: glucose, lactate, choline, tissue oxygen, β-hydroxybutyrate, and reactive-oxygen species.

Example 9: The physiological state monitoring system of any example presented herein, in particular example 1, wherein the multi-analyte sensor probe further includes a counter/reference electrode electrically coupled to the first and second working electrodes.

Example 10: A physiological state monitoring system comprising an implantable multi-analyte sensor probe including a first working electrode configured to generate a first electrical signal indicating an in-tissue concentration of a choline, a second working electrode configured to generate a second electrical signal indicating an in-tissue concentration of oxygen, and a third working electrode configured to generate a third electrical signal indicating an in-tissue concentration of glucose. The system further comprises an electronics module electrically coupled to the sensor probe and including circuitry configured to convert the first electrical signal, the second electrical signal, and the third electrical signal into digital sensor data indicating a choline time-series trace, an oxygen time-series trace, and a glucose time-series trace. The system further comprises a wireless communication interface configured to transmit the digital sensor data, and monitor system control circuitry communicatively coupled to the wireless communication interface and configured to receive the digital sensor data from the electronics module over the wireless communication interface, generate a feature vector that includes one or more of a baseline-subtracted analyte value associated with one or more of the choline time-series trace, the oxygen time-series trace, or the glucose time-series trace, a first-order temporal derivative associated with one or more of the choline time-series trace, the oxygen time-series trace, or the glucose time-series trace, a second-order temporal derivative associated with one or more of the choline time-series trace, the oxygen time-series trace, or the glucose time-series trace, or a ratio between two of the group consisting of: the choline time-series trace, the oxygen time-series trace, and the glucose time-series trace. The monitor control circuitry is further configured to access parameters of a machine-learning classification model trained on historical multi-analyte sensor logs and associated ground-truth physiological states, and use the machine-learning classification model to generate, based on the feature vector, a real-time classification label and an associated confidence value identifying a chemical exposure state.

Example 11: The physiological state monitoring system of any example presented herein, in particular example 10, wherein the machine-learning classification model comprises two or more of a random forest model, a gradient-boosting model, and a feed-forward neural network model.

Example The physiological state monitoring system of any example presented herein, in particular example 11, wherein the real-time classification label is based on outputs from the two or more of the random forest model, the gradient-boosting model, and the feed-forward neural network model.

Example 13: The physiological state monitoring system of any example presented herein, in particular example 11, wherien the monitor system control circuitry is configured to select a single output from among outputs of the two or more of the random forest model, the gradient-boosting model, and the feed-forward neural network model, the real-time classification label corresponding to the selected output.

Example 14: The physiological state monitoring system of any example presented herein, in particular example 10, wherein the monitor system control circuitry is further configured to implement an activation function on the feature vector to introduce nonlinearity to generating the real-time classification label.

Example 15: The physiological state monitoring system of any example presented herein, in particular example 10, wherein the monitor system control circuitry is further configured to normalize at least part of the feature vector.

Example 16: A physiological state monitoring system comprising an implantable multi-analyte sensor probe including a first working electrode configured to generate a first electrical signal indicating a first analyte concentration trace associated with a first analyte, a second working electrode electrically isolated from the first working electrode and configured to generate a second electrical signal indicating a second analyte concentration trace associated with a second analyte, and a third working electrode electrically isolated from the first and second working electrodes and configured to generate a third electrical signal indicating a third analyte concentration trace associated with a third analyte. The system further comprises control circuitry communicatively coupled to the sensor probe and configured to generate rolling median baseline signals for the first electrical signal, the second electrical signal, and the third electrical signal, generate at least one of baseline-subtracted signals or baseline-divided signals for the first analyte concentration trace, the second analyte concentration trace, and the third analyte concentration trace, generate at least one of first-order derivatives or second-order derivatives for the first analyte concentration trace, the second analyte concentration trace, and the third analyte concentration trace, access parameters of a machine-learning classification model trained on historical multi-analyte sensor logs and associated ground-truth physiological states, and use the machine-learning classification model to generate a real-time classification label identifying a physiological state based on the rolling median baseline signals, the at least one of baseline-subtracted signals or baseline-divided signals, and the at least one of first-order derivatives or second-order derivatives.

Example 17: The physiological state monitoring system of any example presented herein, in particular example 16, wherein the control circuitry is further configured to determine pairwise ratios between the first analyte and the second analyte based at least in part on the first analyte concentration trace and the second analyte concentration trace, the first analyte and the third analyte based at least in part on the first analyte concentration trace and the third analyte concentration trace, and the third analyte and the second analyte based at least in part on the third analyte concentration trace and the second analyte concentration trace.

Example 18: The physiological state monitoring system of any example presented herein, in particular example 16, wherein the control circuitry is partially embodied in an electronics module electrically coupled to the sensor probe.

Example 19: The physiological state monitoring system of any example presented herein, in particular example 18, wherein the control circuitry is partially embodied in a mobile computing device configured to communicate with the electronics module over a short-range personal-area-network (WPAN) connection.

Example 20: The physiological state monitoring system of any example presented herein, in particular example 16, wherein the first, second, and third analytes are selected from the group consisting of: glucose, lactate, choline, tissue oxygen, β-hydroxybutyrate, and reactive-oxygen species.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loudspeakers, headphones, pressure transducers, temperature transducers, or using any combination of suitable technologies.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim requires more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A physiological state monitoring system comprising:
an implantable multi-analyte sensor probe including:
a first working electrode configured to generate a first electrical signal proportional to an in-tissue concentration of a first analyte, and
at least one second working electrode electrically isolated from the first working electrode and configured to generate a second electrical signal proportional to a concentration of a second analyte;
an electronics module electrically coupled to the sensor probe and including:
analog front-end circuitry configured to convert the first and second electrical signals into time-stamped digital sensor data, and
a wireless communication interface; and
monitor system control circuitry communicatively coupled to the wireless communication interface and configured to:
receive the digital sensor data from the electronics module over the wireless communication interface;
generate a feature vector that includes one or more of a baseline-subtracted analyte value, a first-order temporal derivative, a second-order temporal derivative, or an inter-analyte ratio based on the digital sensor data;
access parameters of a machine-learning classification model trained on historical multi-analyte sensor logs and associated ground-truth physiological states; and
use the machine-learning classification model to generate, based on the feature vector, a real-time classification label and an associated confidence value identifying a physiological state.

2. The physiological state monitoring system of claim 1, wherein the machine-learning classification model comprises an ensemble including at least two of a random-forest classifier, a gradient-boosted decision-tree classifier, or a feed-forward neural-network classifier.

3. The physiological state monitoring system of claim 1, wherein the monitor system control circuitry is further configured to apply filtering and smoothing to the digital sensor data to remove high-frequency noise and electrode run-in drift prior to generating the feature vector.

4. The physiological state monitoring system of claim 1, wherein the physiological state is selected from a group of physiological states consisting of a plurality of: sleep, exercise, stress, meal ingestion, mustard-agent exposure, organophosphate exposure, opioid exposure, and no exposure.

5. The physiological state monitoring system of claim 1, wherein the multi-analyte sensor probe includes:
a first set of electrode windows exposing the first working electrode; and
a second set of electrode windows exposing the second working electrode;
wherein the first and second sets of electrode windows are interleaved along a central line of the multi-analyte sensor probe.

6. The physiological state monitoring system of claim 5, wherein the multi-analyte sensor probe further includes a third set of electrode windows exposing a third working electrode, the third set of electrode windows being positioned proximal of the first and second sets of electrode windows.

7. The physiological state monitoring system of claim 1, wherein the monitor system control circuitry is further configured to transmit the real-time classification label and the confidence value to at least one of a local user interface or a remote monitoring server.

8. The physiological state monitoring system of claim 1, wherein the first and second analytes are selected from the group consisting of: glucose, lactate, choline, tissue oxygen, β-hydroxybutyrate, and reactive-oxygen species.

9. The physiological state monitoring system of claim 1, wherein the multi-analyte sensor probe further includes a counter/reference electrode electrically coupled to the first and second working electrodes.

10. A physiological state monitoring system comprising:
an implantable multi-analyte sensor probe including:
a first working electrode configured to generate a first electrical signal indicating an in-tissue concentration of a choline,
a second working electrode configured to generate a second electrical signal indicating an in-tissue concentration of oxygen, and
a third working electrode configured to generate a third electrical signal indicating an in-tissue concentration of glucose;
an electronics module electrically coupled to the sensor probe and including:
circuitry configured to convert the first electrical signal, the second electrical signal, and the third electrical signal into digital sensor data indicating a choline time-series trace, an oxygen time-series trace, and a glucose time-series trace; and
a wireless communication interface configured to transmit the digital sensor data; and
monitor system control circuitry communicatively coupled to the wireless communication interface and configured to:
receive the digital sensor data from the electronics module over the wireless communication interface;
generate a feature vector that includes one or more of:
a baseline-subtracted analyte value associated with one or more of the choline time-series trace, the oxygen time-series trace, or the glucose time-series trace;
a first-order temporal derivative associated with one or more of the choline time-series trace, the oxygen time-series trace, or the glucose time-series trace;
a second-order temporal derivative associated with one or more of the choline time-series trace, the oxygen time-series trace, or the glucose time-series trace; or
a ratio between two of the group consisting of: the choline time-series trace, the oxygen time-series trace, and the glucose time-series trace;
access parameters of a machine-learning classification model trained on historical multi-analyte sensor logs and associated ground-truth physiological states; and
use the machine-learning classification model to generate, based on the feature vector, a real-time classification label and an associated confidence value identifying a chemical exposure state.

11. The physiological state monitoring system of claim 10, wherein the machine-learning classification model comprises two or more of:
a random forest model;
a gradient-boosting model; and
a feed-forward neural network model.

12. The physiological state monitoring system of claim 11, wherein the real-time classification label is based on outputs from the two or more of the random forest model, the gradient-boosting model, and the feed-forward neural network model.

13. The physiological state monitoring system of claim 11, wherien the monitor system control circuitry is configured to select a single output from among outputs of the two or more of the random forest model, the gradient-boosting model, and the feed-forward neural network model, the real-time classification label corresponding to the selected output.

14. The physiological state monitoring system of claim 10, wherein the monitor system control circuitry is further configured to implement an activation function on the feature vector to introduce nonlinearity to generating the real-time classification label.

15. The physiological state monitoring system of claim 10, wherein the monitor system control circuitry is further configured to normalize at least part of the feature vector.

16. A physiological state monitoring system comprising:
an implantable multi-analyte sensor probe including:
a first working electrode configured to generate a first electrical signal indicating a first analyte concentration trace associated with a first analyte,
a second working electrode electrically isolated from the first working electrode and configured to generate a second electrical signal indicating a second analyte concentration trace associated with a second analyte, and
a third working electrode electrically isolated from the first and second working electrodes and configured to generate a third electrical signal indicating a third analyte concentration trace associated with a third analyte; and
control circuitry communicatively coupled to the sensor probe and configured to:
generate rolling median baseline signals for the first electrical signal, the second electrical signal, and the third electrical signal;
generate at least one of baseline-subtracted signals or baseline-divided signals for the first analyte concentration trace, the second analyte concentration trace, and the third analyte concentration trace;
generate at least one of first-order derivatives or second-order derivatives for the first analyte concentration trace, the second analyte concentration trace, and the third analyte concentration trace;
access parameters of a machine-learning classification model trained on historical multi-analyte sensor logs and associated ground-truth physiological states; and
use the machine-learning classification model to generate a real-time classification label identifying a physiological state based on:
the rolling median baseline signals;
the at least one of baseline-subtracted signals or baseline-divided signals; and
the at least one of first-order derivatives or second-order derivatives.

17. The physiological state monitoring system of claim 16, wherein the control circuitry is further configured to determine pairwise ratios between:
the first analyte and the second analyte based at least in part on the first analyte concentration trace and the second analyte concentration trace;
the first analyte and the third analyte based at least in part on the first analyte concentration trace and the third analyte concentration trace; and
the third analyte and the second analyte based at least in part on the third analyte concentration trace and the second analyte concentration trace.

18. The physiological state monitoring system of claim 16, wherein the control circuitry is partially embodied in an electronics module electrically coupled to the sensor probe.

19. The physiological state monitoring system of claim 18, wherein the control circuitry is partially embodied in a mobile computing device configured to communicate with the electronics module over a short-range personal-area-network (WPAN) connection.

20. The physiological state monitoring system of claim 16, wherein the first, second, and third analytes are selected from the group consisting of: glucose, lactate, choline, tissue oxygen, β-hydroxybutyrate, and reactive-oxygen species.
